SEQUENCE LISTING (2 sequences)

SEQ ID NO: 1 - DNA, 21 bp, Artificial Sequence
  artifical plasmid
gcgtgcactttttaagggagg

SEQ ID NO: 2 - DNA, 23 bp, Artificial Sequence
  artificial plasmid
cagtatttttccggttgtagccc

(19) United States
(12) Patent Application Publication
(10) Pub. No.: US 2023/0194508 A1
(43) Pub. Date: Jun. 22, 2023

(54) THERMODYNAMICALLY STABILIZED ANTIBODIES FOR DEEP IMMUNOLABELING AND TISSUE IMAGING

(71) Applicant: The Chinese University of Hong Kong, Hong Kong (CN)

(72) Inventors: Ho KO, Hong Kong (CN); Hei Ming LAI, Hong Kong (CN); Yu Him LAU, Hong Kong (CN)

(21) Appl. No.: 17/999,144

(22) PCT Filed: May 21, 2021
(86) PCT No.: PCT/CN2021/095327

§ 371 Date: Nov. 17, 2022

Related U.S. Application Data
(60) Provisional application No. 63/028,022, filed on May 21, 2020.

(51) Int. Cl. G01N 33/533; G01N 33/58

(52) U.S. Cl. CPC G01N 33/533; G01N 33/582

(57) ABSTRACT

The subject invention pertains to methods and compositions to stabilize antibodies for deep immunolabeling and tissue imaging. The antibodies can be stabilized with the addition of antigen-binding fragments of immunoglobulins and cross-linkers and incubated in appropriate buffered conditions.

### CROSS-REFERENCE TO RELATED APPLICATION

This application claims the benefit of U.S. Provisional Patent Application Ser. No. 63/028,022, filed May 21, 2020, which is hereby incorporated by reference in its entirety including any tables, figures, or drawings.

### BACKGROUND OF THE INVENTION

For more than a century, histologists have been studying tissues under the microscope. The methods of histology have not been changed since its advent, which involves the sectioning of tissues into thin micro-meter thick slices before staining and imaging them. Recently, tissue clearing techniques have been developed to obtain three-dimensional views of tissues. The process involves using various chemicals to turn tissues optically transparent and staining the entire tissue block, followed by imaging the cleared tissues in optical sections using laser microscopes. The tissue clearing efficiency and staining penetration depths determine how deep the imaging can be. While remarkably high tissue clearing efficiencies can be achieved, the progress of tissue staining research remained stagnant. In particular, the penetration depths of immunolabeling remained shallow and unpredictable, leading to wasted tissue clearing efforts, and difficulties in applying tissue clearing to human samples in which there are no genetic labeling methods.

Several challenges hampered the development of deep immunostaining methods. Antibodies are unstable, and their interactions with antigens are unpredictable. Based on previous studies, the penetration depths of antibodies negatively correlate with antigen densities, i.e., the denser the distribution, the more antibodies were depleted by superficially located antigens, limiting their deep diffusion into the tissue core. Since antigen densities and the concentration of commercial primary antibodies can vary widely, a general approach to deep immunostaining has been very difficult to develop.

Accordingly a practical, general solution to the challenging problem of deep immunostaining, which empowers modern tissue clearing, is needed.

### BRIEF SUMMARY OF THE INVENTION

Certain embodiments of the subject invention stabilize primary antibodies, particularly at high temperatures. Antigen-binding fragments of immunoglobulins can be added to the primary antibody and then multifunctional cross-linkers can be cross-linked to the antibody complex. In preferred embodiments, the antigen-binding fragments of immunoglobulins are Fab fragments of secondary antibodies or V_{HH} domain fragments of secondary antibodies.

Subsequently, the composition comprising the stabilized primary antibodies provide a general approach to deep immunostaining applicable to all commercial primary antibodies. Antibodies can be diffused into the tissue at high temperatures initially followed by cooling, allowing the antibodies to bind to antigens within the tissue.

In certain embodiments, antibodies can be inhibited from denaturation by cycling temperature to facilitate their diffusion and controlling the antibody-antigen binding kinetics (FIG. 4B). This strategy is termed “thermo-immunohistochemistry with optimized kinetics” or “ThICK”. The methods can use the multifunctional crosslinker polyglycerol 3-polyglycidyl ether (P3PE) for fluorescent protein protection as well as crystallization chaperones (e.g. the antigen-binding fragment (Fab) of antibodies or nanobodies) to stabilize protein conformations for crystallography studies, such as those described by Griffin, L. & Lawson, A. Antibody fragments as tools in crystallography. Clin Exp Immunol 165, 285-291 (2011), which is herein incorporated by reference (FIG. 4C). The crosslinked Ab-Fab complex produced by the methods of the subject invention are termed “synergistically protected polyepoxide-crosslinked Fab-complexed antibody reagents” or “SPEARs”.

In certain embodiments, ThICK and SPEARs can be used with other methods of tissues stabilization or tissue clearing.

### BRIEF DESCRIPTION OF THE DRAWINGS

FIGS. 1A-1B. Three-dimensional immunostaining of a mouse brain without heating the tissue or with heating the tissue to 55° C. with 4% SDS in 1× PBS at a pH of 7.4. (FIG. 1A) Tissue that has been immunostained without heating using primary antibody and Fab fragments of secondary antibody complex. (FIG. 1B) Tissue that has been immunostained with heating using primary antibody and Fab fragments of secondary antibody complex.

FIGS. 2A-2B Three-dimensional immunostaining of a mouse brain without heating the tissue or with heating the tissue to 55° C. with 4% SDS in 1× PBS at a pH of 7.4. (FIG. 2A) Tissue that has been immunostained without heating using primary antibody and Fab fragments of secondary antibody complex in which a cross-linker has been independently mixed into each antibody composition, and then each mixture is combined for immunostaining. (FIG. 2B) Tissue that has been immunostained with heating using primary antibody and Fab fragments of secondary antibody in which a cross-linker has been independently mixed into each antibody composition, and then each mixture is combined.

FIGS. 3A-3B Three-dimensional immunostaining of a mouse brain without heating the tissue or with heating the tissue to 55° C. with 4% SDS in 1× PBS at a pH of 7.4. (FIG. 3A) Tissue that has been immunostained without heating using primary antibody and Fab fragments of secondary antibody that are mixed, and then cross-linkers are added to the antibody composition. (FIG. 3B) Tissue that has been immunostained with heating using primary antibody and Fab fragments of secondary antibody that are mixed, and then cross-linkers are added to the antibody composition.

FIGS. 4A-4M Chemical approach to thermostabilize primary antibodies. FIG. 4A Illustration of antibody (Ab) diffusion to reach deep tissue antigen (Ag) target. For a given Ab at a fixed, limiting concentration applied, the effective diffusion coefficient (D_{eff}) in a tissue sample depends on spatial location of antigens (r) and temperature (T). Ab-Ag binding reactions (with the temperature-dependent dissociation constant of the exothermic forward reaction denoted by K_{d}) deplete Abs, reducing the free Ab concentration (denoted by squared brackets) with increasing depth in the tissue. FIG. 4B Schematic illustration of the general relationships between D_{eff}, K_{d}, percentage of active Abs and T. A higher T increases D_{eff} and favors diffusion down the Ab concentration gradient deeper into the tissue, while Ab-Ag binding reactions are not favored given sufficiently high T (i.e. lowers K_{d}). Abs are readily irreversibly denatured with sufficiently high T (brown solid line), raising T to increase free Ab tissue penetration is viable only if the Abs can be protected from denaturation (brown dotted line). Thermostabilization of Ab can permit a strategy whereby temperature is transiently raised from ambient temperature Tₗ to Tₕ to facilitate diffusion and reduce Ab-Ag binding, thereby increasing free Ab concentration attainable deep in the tissue. When T is lowered afterwards, Ab-Ag binding is favored. FIG. 4C Strategies for stabilizing Abs against permanent heat denaturation. (1) Multifunctional crosslinkers protect Abs from permanent denaturation. (2) The use of secondary Ab antigen-binding fragment (Fab) to further stabilize protein conformation via complexation. FIG. 4D Gel electrophoresis (SDS-PAGE) showing high-molecular weight crosslinked primary Ab-Fab fragment complex. FIG. 4E Tolerance of the crosslinking reaction towards common additives in commercial Abs. FIG. 4F Range of fluorophores applicable on Fabs. FIG. 4G Schematic of the designed ELISA assay variant for functional optimization of SPEARs antigen binding capacity and heat resistance. The fluorescent dyes were replaced with biotinylation to mimic the protected fluorescent Ab-Fab complex utilized for immunostaining. FIG. 4H Optimization of Ab-Fab complex-to-crosslinker ratio in heat protection using our ELISA variant (mean functional SPEARs±S.D. shown, n=4 experimental replicates per group, P=0.0212, Mann-Whitney U test). FIG. 4I Optimization of crosslinking reaction temperature (mean reduction in absorbance±S.D. shown, n=4 experimental replicates per group, P=0.0010, one-way ANOVA). FIG. 4J Optimization of SPEARs heating buffer composition with or without 0.3% Triton X-100 (Tx-100) (mean functional SPEARs±S.D. shown, n=4 experimental replicates per group, P=0.0286, Mann-Whitney U test). FIG. 4K The antigen binding capacity (mean functional SPEARs±S.D.) of the SPEARs before (left panel, 43.2±7.5%) and after (right panel, 98.0±12.9%) functional optimization (P-values shown were obtained by Mann-Whitney U test). FIG. 4L Post-optimization, 15.9±0.8% (mean functional SPEARs±S.D.) SPEARs retained their antigen binding capability after heating for 16 hours at 55° C. FIG. 4M Immunostaining using primary anti-GFAP Ab-Fab fragment complex without crosslinking (left column), separate crosslinking followed by complex formation (middle panels), and crosslinking after complex formation (right panels). Lower panels show results after heating tissue in a denaturant (SDS) for comparison to upper panels before heating. Crosslinking after complex formation is more effective in protection from denaturation than separately crosslinking Ab and Fab fragments.

FIGS. 5A-5M Development and applications of deep immunostaining using thermostabilized primary antibody-Fab complex. FIG. 5A Tolerance of SPEARs to the duration of ThICK staining at 55° C. Upper panels: x-z view of mouse spinal cords ThICK-stained with ChAT SPEARs. Scale bar: 50 μm. Lower panels, example cells from different depths. Scale bar: 10 μm. Intensity scale bar: pixel intensity. FIG. 5B Homogeneity of pixel intensity mean (left panel), variability (S.D.) (middle panel) and signal-to-noise ratio (SNR) (right panel) across depth positively correlates with ThICK staining duration. The 72-hours experiment was excluded from SNR analysis due to the absence of an appropriate background in the imaged tissue volume. FIG. 5C Compatibility with endogenous fluorescence with short heating for formaldehyde-fixed samples (left panels) and longer heating (up to 16 hr) for SHIELD-protected samples (right panels). FIG. 5D Range of antibody-antigen pairs applicable. Colors represent the fluorescent dyes used for imaging (green: AlexaFluor-488, red: AlexaFluor-593, cyan: AlexaFluor-647). FIG. 5E Optimization of staining by adjusting ThICK staining buffer composition with respect to SPEARs intravascular precipitates per imaged tissue volume. After confirming 1 M GnCl as the optimal ThICK-staining component, the experiment was repeated two more times along with control (0.3% Tx). Error bars depict S.D.s for these groups. P=0.0216, Mann-Whitney U test. FIG. 5F Immunostaining with ChAT SPEARs before and after ThICK staining buffer optimization. Insets: enlarged views of representative cells in white boxed areas. Pixel intensity color scale: same as in FIG. 5B. FIG. 5G Principle of pyridine (py)-catalyzed P3PE crosslinking reaction. The pyridinium intermediate acts as a good leaving group for the S_{N}2 reaction. FIG. 5H Higher concentration of py is associated with more conversion of precursor to product. FIG. 5I Addition of 61.8 mM py showed faster crosslinking than non-catalyzed control by SDS-PAGE. FIG. 5J Schematic of functional assay based on hot-start PCR for testing pyridine-catalyzed synthesized SPEARs (SPEAR^{py}) and agarose gel analysis of so-formed PCR product in the lower panel. FIG. 5K Quantified functional activity of Taq SPEAR versus Taq SPEAR^{py} on inhibition of formation of PCR product, SPEARs were used directly after synthesis versus pre-heated at 55° C. for 16 hours. Experiment was repeated 6 times independently for each group, error bars depict S.D.s for these groups. n.s. not significant, *** P≤0.001, Tukey's multiple comparison test. FIG. 5L ChAT SPEARs formed in the presence of 61.8 mM py results in better staining quality than SPEARs produced without py. Left panel: illustrative images obtained from staining with ChAT SPEARs. Right panel: signal-to-background ratios along the axes of representative cells (in white rectangles) from the left panel. Lighter lines represent normalized intensity profiles of individual cells. Solid lines represent the mean of 5 cells. Shaded regions: S.D. for each group. FIG. 5M Application of ChAT SPEARs (red, obtained by 61.8 mM py-catalyzed crosslinking) for optimized ThICK staining in SHIELD-protected sample with endogenous neuronal GCaMP6f (green). Precipitates can be easily identified and are digitally removable (white).

FIGS. 6A-6E Application of SPEARs to ThICK-staining of human brain tissue and the whole mouse brain. FIG. 6A Illustrated protocol used for a 5 mm-thick human brainstem block ThICK-staining with TH SPEAR^{py}. Timeline (in hours) were drawn to scale. FIG. 6B Overview of a tiled Z-stack of the imaged 5 mm-thick human pons block containing the locus coeruleus. FIG. 6C Magnified x-z view of the white boxed area in FIG. 6B demonstrating 700 um-deep TH-positive neurons. FIG. 6D Conventional immunostaining with TH antibodies in the same region of a human brain tissue (left) compared with the TH SPEAR ThICK-stained human tissue in FIG. 6B (right). Annotated are segmented TH-positive cell bodies with their depth intensity-coded according to the displayed color bar. FIG. 6E shows the quantified distribution of the segmented cell bodies in FIG. 6D with distance from nearest tissue surface. Difference in mean by unpaired two-sample t-test with indicated P=0.0001.

FIGS. 7A-7C Establishment of P3PE-crosslinked IgG-Fab complex electrophoretic patterns and initial optimization of reaction condition for yields. FIG. 7A Reducing and FIG. 7B non-reducing SDS-NuPAGE analysis of P3PE-crosslinked IgGs, Fabs and their complexes under various conditions and their electrophoretic patterns. FIG. 7C Time course of P3PE-crosslinking of IgG-Fab complexes. The tested reaction conditions are listed on the right.

FIGS. 8A-8C Testing tolerance of P3PE-crosslinking reaction towards common additives in commercially supplied antibodies using reducing SDS-PAGE. FIG. 8A Screening for additives that inhibit P3PE-crosslinking of IgG-Fab complexes. FIG. 8B and FIG. 8C titration of Tris (FIG. 8B) and BSA (FIG. 8C) and their effects on P3PE-crosslinking reaction. The tested reaction conditions are listed on the right.

FIG. 9 Performance of the ELISA variant for functional optimization of SPEARs. The absorbance response of ABTS is linear over four orders of antigen dilution. The line of best fit on linear regression (black) and its equation are shown. Dotted lines: 95% confidence interval of regression.

FIG. 10 Optimization of ThICK staining protocol. Additives were added in various incubation steps while vascular precipitation of SPEARs was globally quantified for each imaged tissue stack and normalized against the imaged tissue volume (see Methods). The optimization was performed iteratively for four rounds (grouped in colors). For all experiments, permeabilization was performed at 37° C. for 1 day, ThICK staining was performed at 55° C. for 16 hours, and post-washing was performed at RT for 1 day. Abbreviations: BSA, bovine serum albumin; GnCl, guanidinium chloride; PBST, 1× phosphate buffered saline with 0.3% v/v Triton X-100; TMAO, trimethylamine oxide; Tx, Triton X-100; SDC, sodium deoxycholate; SDS, sodium dodecyl sulfate.

FIGS. 11A-11B Post-imaging removal of intravascular SPEAR precipitates. FIG. 11A Approach for segmenting and removing intravascular SPEAR precipitates using commercial software (Imaris, see Methods). FIG. 11B Removal of VIP SPEAR intravascular precipitates after one round of image processing.

FIGS. 12A-12F Development of a catalyst for P3PE-crosslinking of amine-containing proteins. FIG. 12A Catalyst conception based on the use of a nucleophile (Nu) that can result in an intermediate with a good leaving group, and/or the use of Lewis acids (LeA) to facilitate the nucleophilic ring opening. FIG. 12B Lewis acids compatible with our reaction condition are lithium and ammonium ions. FIG. 12C Nucleophiles compatible with our reaction condition are those with high nucleophilicities and low basicity in a protic solvent environment, including pyridines, sterically hindered trisubstituted amines, and imidazoles. However, aliphatic amines and imidazoles can lead to failed crosslinking due to side reactions. FIGS. 12D-12F Reducing SDS-PAGE for screening and confirming catalytic activities. The tested reaction conditions are listed on the right. FIG. 12D Screened catalyst candidates chosen based on rationales described in FIGS. 12A-12C using reducing SDS-PAGE. The tested reaction conditions are listed on the right. FIG. 12E Confirmation of catalytic and non-catalytic effect of pyridine and lithium on SPEARs formation, respectively. FIG. 12F Exploration of pyridine's catalytic effect under various conditions.

FIGS. 13A-13D Optimization and characterization of pyridine-catalyzed formation of SPEARs. FIGS. 13A-13C Reducing SDS-PAGE for optimization and characterization of pyridine-catalyzed P3PE-crosslinking reaction. The tested reaction conditions are listed on the right. FIG. 13A Titration of pyridine concentration in the reaction mixture. FIG. 13B Effect of antibody-Fab complex concentration and P3PE concentration on the overall yield of SPEARs. FIG. 13C Time course of pyridine-catalyzed P3PE-crosslinking reaction. FIG. 13D Comparison of ThICK staining quality with ChAT SPEARs and calretinin (Calret) SPEARs with and without the use of pyridine. Color scale bar: pixel intensity.

### DETAILED DISCLOSURE OF THE INVENTION

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, a “primary antibody” is an antibody that binds to proteins or antigens directly.

As used herein, a “secondary antibody” is an antibody that binds to another (primary) antibody. In certain embodiments, the primary antibody is already bound to an antigen or protein.

As used herein, “immunolabeling” is a process to detect and localize an antigen to a particular site within a cell, tissue, or organ. Immunolabeling can comprise direct immunolabeling in which the antibody that binds directly to the antigen is labeled. Or, immunolabeling can comprise indirect immunolabeling in which a secondary antibody is labeled. To visualize the immunolabeling, various methods are known in the art. Some methods include fluorescence, chemiluminescence, chromogenic, or colorimetric.

In certain embodiments, the immunolabels can include fluorescent labels and quencher labels. Exemplary fluorescent labels include a quantum dot or a fluorophore. Examples of fluorescence labels for use in this method includes fluorescein, 6-FAM™ (Applied Biosystems, Carlsbad, Calif.), TET™ (Applied Biosystems, Carlsbad, Calif.), VIC™ (Applied Biosystems, Carlsbad, Calif), MAX, HEX™ (Applied Biosystems, Carlsbad, Calif), TYE™ (ThermoFisher Scientific, Waltham, Mass.), TYE665, TYE705, TEX, JOE, Cy™ (Amersham Biosciences, Piscataway, N.J.) dyes (Cy2, Cy3, Cy3B, Cy3.5, Cy5, Cy5.5, Cy7), Texas Red® (Molecular Probes, Inc., Eugene, Oreg.), Texas Red-X, AlexaFluor® (Molecular Probes, Inc., Eugene, Oreg.) dyes (AlexaFluor 350, AlexaFluor 405, AlexaFluor 430, AlexaFluor 488, AlexaFluor 500, AlexaFluor 532, AlexaFluor 546, AlexaFluor 568, AlexaFluor 594, AlexaFluor 610, AlexaFluor 633, AlexaFluor 647, AlexaFluor 660, AlexaFluor 680, AlexaFluor 700, AlexaFluor 750), DyLight™ (ThermoFisher Scientific, Waltham, Mass.) dyes (DyLight 350, DyLight 405, DyLight 488, DyLight 549, DyLight 594, DyLight 633, DyLight 649, DyLight 755), ATTO™ (ATTO-TEC GmbH, Siegen, Germany) dyes (ATTO 390, ATTO 425, ATTO 465, ATTO 488, ATTO 495, ATTO 520, ATTO 532, ATTO 550, ATTO 565, ATTO Rho101, ATTO 590, ATTO 594, ATTO 610, ATTO 620, ATTO 633, ATTO 635, ATTO 637, ATTO 647, ATTO 647N, ATTO 655, ATTO 665, ATTO 680, ATTO 700, ATTO 725, ATTO 740), BODIPY® (Molecular Probes, Inc., Eugene, Oreg.) dyes (BODIPY FL, BODIPY R6G, BODIPY TMR, BOPDIPY 530/550, BODIPY 558/568, BODIPY 564/570, BODIPY 576/589, BODIPY 581/591, BODIPY 630/650, BODIPY 650/665), HiLyte Fluor™ (AnaSpec, Fremont, Calif.) dyes (HiLyte Fluor 488, HiLyte Fluor 555, HiLyte Fluor 594, HiLyte Fluor 647, HiLyte Fluor 680, HiLyte Fluor 750), AMCA, AMCA-S, Cascade® Blue (Molecular Probes, Inc., Eugene, Oreg.), Cascade Yellow, Coumarin, Hydroxycoumarin, Rhodamine Green™-X (Molecular Probes, Inc., Eugene, Oreg.), Rhodamine Red™-X (Molecular Probes, Inc., Eugene, Oreg.), Rhodamine 6G, TMR, TAMRA™ (Applied Biosystems, Carlsbad, Calif.), 5-TAMRA, ROX™ (Applied Biosystems, Carlsbad, Calif.), Oregon Green® (Life Technologies, Grand Island, N.Y.), Oregon Green 500, IRDye® 700 (Li-Cor Biosciences, Lincoln, Nebr.), IRDye 800, WeIIRED D2, WeIIRED D3, WeIIRED D4, and Lightcycler® 640 (Roche Diagnostics GmbH, Mannheim, Germany). In some embodiments, bright fluorophores with extinction coefficients >50,000 M⁻¹ cm⁻¹ and appropriate spectral matching with the fluorescence detection channels can be used.

In certain embodiments, a fluorescently labeled protein is included in a reaction mixture and a fluorescently labeled reaction product is produced. Fluorophores used as labels to generate a fluorescently labeled protein included in embodiments of methods and compositions of the present invention can be any of numerous fluorophores including, but not limited to, 4-acetamido-4′-isothiocyanatostilbene-2,2′ disulfonic acid; acridine and derivatives such as acridine and acridine isothiocyanate; 4-amino-N-[3-vinylsulfonyl)phenyl]naphthalimide-3,5 disulfonate, Lucifer Yellow VS; N-(4-anilino-1-naphthyl)maleimide; anthranilamide, Brilliant Yellow; BIODIPY fluorophores (4,4-difluoro-4-bora-3a,4a-diaza-s-indacenes); coumarin and derivatives such as coumarin, 7-amino-4-methylcoumarin (AMC, Coumarin 120), 7-amino-4-trifluoromethylcoumarin (Coumaran 151); cyanosine; DAPDXYL sulfonyl chloride; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-4′-dimethylaminophenylazo)benzoic acid (DABCYL); 4-dimethylaminophenylazophenyl-4′-isothiocyanate (DABITC); EDANS (5-[(2-aminoethyl)amino]naphthalene-1-sulfonic acid), eosin and derivatives such as eosin isothiocyanate; erythrosin and derivatives such as erythrosin B and erythrosin isothiocyanate; ethidium such as ethidium bromide; fluorescein and derivatives such as 5-carboxyfluorescein (FAM), hexachlorofluorescenin, dichlorotriazin-2-yl)aminofluorescein (DTAF), 2′,7′-dimethoxy-4′,5′-dichloro-6-carboxyfluorescein (JOE) and fluorescein isothiocyanate (FITC); fluorescamine; green fluorescent protein and derivatives such as EBFP, EBFP2, ECFP, and YFP; IAEDANS (5-({2-[(iodoacetyl)amino]ethyl} amino)naphthalene-1-sulfonic acid), Malachite Green isothiocyanate; 4-methylumbelliferone; orthocresolphthalein; nitrotyrosine; pararosaniline; Phenol Red; B-phycoerytnin; o-phthaldialdehyde; pyrene and derivatives such as pyrene butyrate, 1-pyrenesulfonyl chloride and succinimidyl 1-pyrene butyrate; QSY 7; QSY 9; Reactive Red 4 (Cibacron® Brilliant Red 3B-A); rhodamine and derivatives such as 6-carboxy-X-rhodamine (ROX), 6-carboxyrhodamine (Rhodamine 6G), rhodamine isothiocyanate, lissamine rhodamine B sulfonyl chloride, rhodamine B, rhodamine 123, sulforhodamine B, sulforhodamine 101 and sulfonyl chloride derivative of sulforhodamine 101 (Texas Red); N,N,N′,N-tetramethyl-carboxyrhodamine (TAMRA); tetramethyl rhodamine; tetramethyl rhodamine isothiocyanate (TRITC); riboflavin; rosolic acid and terbium chelate derivatives.

Exemplary quencher labels include a fluorophore, a quantum dot, a metal nanoparticle, and other related labels. Suitable quenchers include Black Hole Quencher®-1 (Biosearch Technologies, Novato, Calif.), BHQ-2, Dabcyl, Iowa Black® FQ (Integrated DNA Technologies, Coralville, Iowa), IowaBlack RQ, QXL™ (AnaSpec, Fremont, Calif.), QSY 7, QSY 9, QSY 21, QSY 35, IRDye QC, BBQ-650, Atto 540Q, Atto 575Q, Atto 575Q, MGB 3′ CDPI3, and MGB-5′ CDPI3. Fluorescence is quenched when the fluorescence emitted from the fluorophore is detectably reduced, such as reduced by 10%, 20%, 30%, 40%, 50%, 60%, 70%, 80%, 90%, 95%, 99% or more. Numerous fluorophore quenchers are known in the art, including, dabcyl; sulfonyl chlorides such as dansyl chloride; and Black Hole Quenchers BHQ-1, BHQ-2 and BHQ-3.

In certain embodiments of the subject invention, a primary antibody can be stabilized for ensuing use deep immunolabeling and tissue imaging, preferably thermally stabilized. In other embodiments, the primary antibody can be stabilized in various other conditions, such as, for example, acidic, basic, ionic, or in solutions with various solvents and/or chemical additives. The primary antibody can be a commercially produced antibody or an antibody produced by one skilled in the art. The primary antibody can be used for immunolabeling, tissue imaging, detecting proteins, quantifying proteins, or any other related process.

In certain embodiments, the primary antibody is combined with antigen-binding fragments of immunoglobulins and cross-linkers. The primary antibody can be combined with the antigen binding fragments of immunoglobulins and cross-linkers concurrently or initially combined with the cross-linkers and then the antigen binding fragments of immunoglobulins. In preferred embodiments, the primary antibody is combined with the antigen binding fragments of immunoglobulins and then the cross-linkers. In certain embodiment, the mixture of the primary antibody with the antigen-binding fragments of immunoglobulins and/or cross-linkers is further comprised of a buffer. In preferred embodiments, the buffer is 0.1× 0.5×, 1×, 2.5×, 5×, or 10× phosphate-buffered saline (PBS) or phosphate-buffered saline and 0.1% Tween 20 detergent (PBST) or 0.01M, 0.025M, 0.05M, 0.075M, 0.1M, 0.25M, 0.5M, 0.75M, or 1M sodium carbonate. The buffer can be present before the addition of either the cross-linkers or the antigen binding fragments of immunoglobulins to the primary antibody, concurrently with the addition of either the cross-linkers or antigen binding fragments of immunoglobulins to the primary antibody, or after the addition of the cross-linkers and/or antigen binding fragments of immunoglobulins.

In certain embodiments, the immunoglobulins from which the antigen-binding fragments of immunoglobulins are derived are secondary immunoglobulins. In preferred embodiments, the antigen-binding fragments of immunoglobulins can be Fab fragments of secondary antibodies. In certain embodiments, the Fab fragments originate from donkey or goat, but other organisms are envisioned, including mammals, such as, for example, mouse, sheep, llama, horse, cat, cow, dog and rabbit or birds, such as, for example, chicken. In other embodiments, the antigen-binding fragments of immunoglobulins are V_{HH} domain fragments of secondary antibodies. In preferred embodiments, the V_{HH} domain fragments of secondary antibodies are derived from organisms in the biological family Camelidae. In certain embodiments, the antigen-binding fragments of immunoglobulins are raised to target the primary antibody's host species' immunoglobulins.

In certain embodiments, the antigen-binding fragments of immunoglobulins are incubated with the primary antibody of interest at a molar ratio of about 1:5 to about 10:1, about 1:2 to about 5:1, or, preferably about 1:1 to about 3:1 (antigen binding fragments of immunoglobulins: primary antibody) for at least 1 minute, 2 minutes, 5 minutes, 10 minutes, 15 minutes, 20 minutes, 30 minutes, or greater at about 4° C. to about 37° C. about 10° C. to about 30° C., or at about room temperature, with an amount of primary antibody at a final concentration of at least 0.01, 0.1, 0.25, 0.5, 0.75, 1, 2, 5, 10 mg/ml, or greater.

In certain embodiments, the cross-linker is a homo-multifunctional cross-linker. In preferred embodiments, the homo-multifunctional cross-linker is Polyglycerol-3-polyglycidyl ether (P3PE); however, other homo-multifunctional cross-linkers are envisioned such as, for example, 4-Arm PEG-SCM, MW 2k; 4-Arm PEG-SC, MW 2k; 4-Arm PEG-SG, MW 2k; 4-Arm PEG-SS, MW 2k; 4-Arm PEG-SAS, MW 2k; GAS-PEG-GAS, MW 2k; SAS-PEG-SAS, MW 2k; SG-PEG-SG, MW 2k; 4arm PEG Succinimidyl Glutaramide; 4arm PEG, 3arm Methoxy, 1arm Succinimidyl Carboxymethyl Ester; 8arm PEG Succinimidyl Succinate (tripentaerythritol); BS (PEG)5; BS (PEG)9; tris-Succinimidyl aminotriacetate; tris-Succinimidyl (6-aminocaproyl)aminotriacetate; or tetrakis-(N-succinimidylcarboxypropyl)pentaerythritol. In certain embodiments, before the cross-linker is added to the primary antibody mixture, it is diluted to about a 1% to about 50%, or about a 5% to about a 30%, or about a 10% to about a 20% v/v solution in water and vortexed for at least 15 seconds, 30 seconds, 1 minute, 2 minutes, or greater at about room temperature. The cross-linker solution can then be centrifuged, and the supernatant resulting from the centrifugation can be used in the primary antibody reaction mixture at about a 1:1, 1:2, 1:3, 1:4, 1:5, 1:6, 1:7, 1:8, 1:9 or 1:10 dilution (cross-linker: antibody reaction mixture). In preferred embodiments, the dilution is 1:5.

In certain embodiments, a cross-linking reaction using the cross-linker and at least one antibody is performed for at least 2, 4, 8, 12, 24, 36, 48, 72 hours, or greater. In preferred embodiments, the primary antibody is mixed with the antigen-binding fragments of immunoglobulins before the cross-linking reaction. After cross-linking, a quenching reagent can be used to quench the cross-linking. The quenching reagent can be, for example, an acid, a strong base (e.g. 0.1-3M sodium hydroxide, Tris base at pH 6-9), ammonium chloride (0.1-2M) in 1× Phosphate buffered saline (pH 7.4), 0.1M sodium bicarbonate buffer (pH 10.0), amines (e.g. lysine) in various concentrations (0.1-1M), or sodium azide 0.01-0.1% w/v. The antibody mixture can now be used for immunolabeling, immunostaining, deep tissue imaging, or other related process. Additionally, the antibody mixture may be purified, diluted, or processed in any other manner that does not disrupt the cross-linked antibody complex.

In certain embodiments, the cross-linking reaction using the cross-linker and at least one antibody can optionally contain a catalyzing agent upon initiation of the cross-linking reaction at a concentration of about 1 to about 1000 mM, about 1 to about 500 mM, about 1 to about 250 mM, about 1 to about 128 mM, or about 61.8 mM, about 62 mM , about 63 mM , about 64 mM , about 65 mM , about 61 mM, or about 60 mM. The catalyzing agent can be pyridine or related derivatives, such as, for example, niacin, nicotinamide, isonicotinoylhydrazine, nicotine, N-methylnicotinamide, strychnine, and vitamin B6.

In certain embodiments, the antibody complex product generated using the primary antibody, the antigen-binding region of an immunoglobulin, and the cross-linker can be used for immunolabeling. The immunolabeling can be 3D immunolabeling in biological tissues, cells, or organs. During immunolabeling, the tissues, cells, or organs can be heated to at least 30° C., 37° C., 40° C., 45° C., 50° C., 55° C., 60° C., 65° C. or greater. In preferred embodiments, the tissue can be heated to 55° C. After heating, the tissues, cells, or organs and composition of the subject invention can be cooled to room temperature for at least 10 min, 20 min, 30 min, 45 min, 1 hour, 2 hours, 4 hours, or greater. Additional chemicals can be added to the antibody mixture and the biological cells, tissues, or organs that are being immunolabeled. In certain embodiments, the chemical is sodium dodecyl sulfate (SDS). SDS can be present at a concentration of at least 0.1%, 1%, 2%, 4%, 6%, 8%, 10%, or greater. Other chemicals can be used in place of SDS, including detergents, radioiodinated contrasts, denaturants, or blocking agents. Examples of detergents that can be used in a concentration of at least 0.1% to about 10% or greater include, cationic detergents (e.g. cetyltrimethylammonium bromide), anionic detergents (e.g. sodium deoxycholate, at 10% w/v), neutral detergents (e.g. 1,2-hexanediol, Triton X-100 (at 0.3% v/v), Tween 20), or zwitterionic detergents (e.g. CHAPS). Examples of radioiodinated contrasts are iopromide and iohexol. Examples of blocking agents can include bovine serum albumin (at 1-5% w/v, 1%), glycine (0.1-2M, 0.6M), or normal donkey serum (at 1-10% v/v, 3%). Examples of denaturants to be included throughout the staining process include Guanidinium chloride, urea, and trimethylamine oxide. The denaturants can be at a concentration of about 0.1 M to about 10 M or about 1 M. Buffers can be used in the immunolabeling process, such as, for example, 0.1×, 0.5×, 1×, 2.5×, 5×, or 10× PBS. In certain embodiments, the pH at which the immunolabeling is performed is at least 5, 6, 7, 7.2, 7.4, 7.6, 7.8, 8, or 9. In preferred embodiments, the pH is 7.4. Additional modifications to chemicals, buffers, temperature and pH are envisioned. Immunolabeling is well known in the art to be dependent on a variety of factors, such as, for example, cell, tissue, or organ type; type of immunolabeling, such as, for example, immunolabeling with fluorescence detection, immunolabeling with DNA barcoding, and fluorescent DNA readout; and elapsed time for the immunolabeling to be performed.

### Materials and Methods

### Chemicals and Reagents

All chemicals were stored at temperatures as recommended by their vendors, protected from light, and used without further purification. The secondary antibodies Fab fragments used were Alexa Fluor 594-conjugated donkey anti-goat IgG Fab fragments (Cat. no. 705-547-003, Jackson ImmunoResearch, West Grove, Pa.), unconjugated donkey anti-mouse IgG Fab fragments (Cat. no. 715-007-003, Jackson ImmunoResearch), Alexa Fluor 488-conjugated donkey anti-mouse IgG Fab fragments (Cat. no. 715-547-003, Jackson ImmunoResearch), Alexa Fluor 594-conjugated donkey anti-mouse IgG Fab fragments (Cat. no. 715-587-003, Jackson ImmunoResearch), Alexa Fluor 647-conjugated donkey anti-mouse IgG Fab fragments (Cat. no. 715-607-003, Jackson ImmunoResearch), unconjugated donkey anti-rabbit IgG Fab fragments (Cat. no. 711-007-003 Jackson ImmunoResearch), Alexa Fluor 488-conjugated donkey anti-rabbit IgG Fab fragments (Cat. no. 711-547-003, Jackson ImmunoResearch), Alexa Fluor 594-conjugated donkey anti-rabbit IgG Fab fragments (Cat. no. 711-587-003, Jackson ImmunoResearch), Alexa Fluor 647-conjugated donkey anti-rabbit IgG Fab fragments (Cat. no. 711-607-003, Jackson ImmunoResearch), and Alexa Fluor 488-conjugated goat anti-rat IgG Fab fragments (Cat. no. 112-547-003, Jackson ImmunoResearch). The lyophilized Fab fragments were reconstituted using distilled water to a concentration of 1 mg/ml and stored at 4° C. in aliquots.

### Mouse Brain Tissue

All experimental procedures were approved in advance by the Animal Research Ethical Committee of the Chinese University of Hong Kong and were carried out in accordance with the Guide for the Care and Use of Laboratory Animals. C57BL/6 and Thy1-GCaMP6f transgenic adult mice of at least 2 months old were used. Formaldehyde-fixed and SHIELD-protected brain tissues were harvested as previously described in Park, Y.-G. et al. Protection of tissue physicochemical properties using polyfunctional crosslinkers. Nat Biotechnol 37, 73-83 (2019), which is hereby incorporated by reference. SHIELD protection is preferably for samples labeled with fluorescent proteins. After adequate washings with PBST, tissues were stored at 4° C. in 1× PBS until use.

### Chemical Stabilization of Antibody-Fab Fragment Complex

IgGs and their corresponding secondary Fab fragments were first reconstituted or diluted to a stock solution of 1 mg/ml with distilled water. 1 ul of the stock IgG solution was then thoroughly mixed with 1 μl of the stock Fab fragment solution and incubated at room temperature in a 0.2 ml PCR tube for 10 minutes for complex formation. During this time, 200 μl of P3PE (Huntsman, Erisys GE-38, The Woodlands, Texas) was pipetted into a 1.5 ml Eppendorf tube using cut tips and reverse pipetting technique. 800 μl of distilled water was then added and the tube was tightly capped and vigorously vortexed for 1 minute where the mixture would become a homogeneous milky emulsion. The tube was then centrifuged at 15,000×g for 3 minutes at room temperature (RT) and allowed to sit at RT for not longer than an hour. 1 μl of 1 M sodium carbonate pH 10 buffer followed by 5 μl of water were then added to the formed IgG-Fab complex and thoroughly mixed. This is followed by adding 2 μl of the prepared P3PE supernatant, and the tube was immediately vortexed. Using a thermocycler, the 10 μl reaction mixture was then reacted at 37° C. for a certain time period followed by cooling to 4° C. and kept for not more than 24 hours until further use. The reaction can be scaled up to 100 μl each time per PCR tube.

SPEARs Synthesis from Commercially Available Primary Antibodies

Primary antibodies were reconstituted in 1× PBS with 0.1% w/v sodium azide to 1 μg/μl if lyophilized. The constituents of the storage buffer were reviewed for presence of any additives (except BSA) containing primary amine groups. If the storage buffer contains >0.1 M Tris, the antibodies were buffer exchanged to 1× PBS using ultracentrifugal filters with molecular weight cut-off of 50 kDa (Amicon Ultra-0.5 centrifugal filter unit, Cat. no. UFC505008, Millipore, Burlington, Mass.). Purified antibodies in serum are preferred, as non-specific IgGs would consume the Fab fragments. 2 μl of 0.05 μg/μl antibody complexed with 2 μl of 1 μg/μl Fab fragment performed as satisfactorily as 2 μl of 1 μg/μl antibody, although using a larger amount of antibody may help to further boost signal.

SPEARs were freshly synthesized 1 day prior to staining. 2 μl of the primary antibody and 1 μl of the corresponding Fab fragment at 2 μg/μl were thoroughly mixed and incubated at room temperature for 10 minutes to form the Ab-Fab complex. During this time, 200 μl of P3PE was pipetted into a 1.5 ml Eppendorf tube using cut 1000-μl tips and the reverse pipetting technique. 800 μl of distilled water was then added and the tube was tightly capped and vigorously vortexed for 1 minute where the mixture would become a homogeneous milky emulsion. The tube was then centrifuged at 15,000×g for 3 minutes at room temperature and allowed to sit at room temperature for not longer than an hour. To form the IgG-Fab complex, 1 μl of 1 M sodium carbonate pH 10 buffer followed by 4 μl of water were then added and thoroughly mixed. This is followed by 1 μl of the freshly prepared P3PE supernatant, and the tube was rigorously vortexed. Using a thermocycler, the 10 μl reaction mixture was then reacted at 13° C. for 16 hours followed by cooling to 4° C. and kept for not more than 24 hours until further use. The reaction can be scaled up to 100 μl each time per PCR tube.

### OPTIClear2

OPTIClear2 is an improved version of the original hydrophilic optical clearing solution OPTIClear (OPTIClear is described in Lai, H. et al. Next generation histology methods for three-dimensional imaging of fresh and archival human brain tissues. Nat Commun 9, 1066 (2018), which is hereby incorporated by reference). OPTIClear2 features easier preparation, faster and better optical clearing (although OPTIClear is also compatible with SPEARs and ThICK staining). OPTIClear2 is comprised of 20% v/v 1-(3-aminopropyl)imidazole (Cat. no. A14169, Alfa Aesar, Haverhill, Mass.), 25% w/v 2,2′-thiodiethanol (Cat. no. 166782, Sigma-Aldrich, St. Louis, Mo.), and 32% w/v iopromide (Ultravist 370, Bayer, Leverkusen, Germany) without further pH adjustments. OPTIClear is comprised of 20% w/v N-methylglucamine (Cat. no. M2004, Sigma-Aldrich), 25% w/v 2,2′-thiodiethanol, and 32% w/v iohexol (Nycodenz, Cat. no. 1002424, Progen Heidelberg, Germany), with pH adjusted to 7-8 using concentrated hydrochloric acid.

### Confocal Microscopy

Unless otherwise specified, confocal microscopy was performed using a Leica TCS SP8 confocal microscope. Excitation laser wavelengths used were 488 nm, 514 nm, 561 nm and 649 nm. Detection was done using GaAsP PMTs through an HC PL APO×10/0.40 CS2 (FWD 2.2 mm) or an HC PL APO×20/0.75 CS2 (FWD 0.62 mm) objective. All imaging parameters were controlled for each set of experiments.

### Image Processing and Digital Removal of Intravascular SPEAR Precipitates

To digitally remove precipitate signals, an acquired multi-channel confocal z-stack image in .lif format was first imported into Fiji (ImageJ) and exported in .tiff format, as described in Schindelin, J. et al. Fiji: an open-source platform for biological-image analysis. Nat Methods 9, 676-82 (2012), which is herein incorporated by reference. The tiff image was then imported into Imaris (v9, Bitplane, Zürich, Switzerland). A surface was then created based on the SPEARs staining channel, with local contrast settings, surface detail at 1.0 μm and maximal object diameter at 10.0 μm. The created surfaces were then filtered based on their specificities with regards to the vasculature and further edited manually. The created surfaces were then used to mask and set the intra-surface voxels intensities to zero.

### Image Analysis for Staining Homogeneity Across Z-Depth

ROIs of positive staining and background regions of ChAT SPEARs-stained mouse spinal cord sections were manually inspected and defined. The pixel intensities for the ROIs of each image slice were then profiled through the z-depth, with their means, standard deviations, and SNR calculated using a custom-written MATLAB program (R2018b, MathWorks, Portola Valley, Calif.). For each image, the SNR is defined as the ratio of summed squared pixel intensity of ROIs of positive staining to that of background regions (r), expressed in decibel (dB) (i.e. SNR=10 log₁₀(r)).

### Image Analysis for Quantification of Intravascular SPEAR Precipitates

Definition and surface masking of intravascular SPEAR precipitates was performed as above for their digital removal using Imaris. The total tissue volume was similarly measured with surface rendering and masking except with surface detail at 5.0 μm and without local contrast and background subtraction. Intravascular SPEAR precipitates volumes and total tissue volumes were automatically quantified based on the generated surfaces in Imaris and exported for analysis.

### Polyacrylamide Gel Electrophoresis and Densitometry

IgGs were complexed with their respective fluorescently labeled secondary antibody Fab fragments and crosslinked under various conditions as described in the figures at 10 μl reaction scale. The completed reaction mixture was then mixed with 3.5 μl of 4× NuPAGE LDS sample loading buffer (Invitrogen NP007, Carlsbad, Calif.) and 0.5 μl of beta-mercaptoethanol, heated to 95° C. for 10 minutes and cooled to room temperature. The samples were then loaded onto 1 mm-thick 10% SDS-polyacrylamide gels or 10% NuPAGE Bis-Tris gels (Cat. no. NP0301BOX, Invitrogen) and ran at a constant voltage of 90-120 V until the loading dye front reached the bottom of the gel. The gels were stained in InstantBlue Protein Stain (Cat. no. ISB01L, Expedeon, Cambridge, United Kingdom) overnight at room temperature with gentle shaking. Brightfield gel images were taken with a smartphone camera under ambient white light while fluorescence gel images were taken with a BioRad (Hercules, Calif.) Gel Doc EZ System with automatic exposure. The obtained gel band intensities were measured using Fiji with manually defined ROIs, the quantification procedures have been kept constant for all bands within the same set of experiments.

Functional Optimization of SPEAR Antigen Binding Capacity with an ELISA Variant

96-well ELISA plates (Nunc MaxiSorp flat-bottom plates, Cat. no. 44-2404-21, ThermoFisher Scientific) were coated with a 10 mg/ml stock solution of NeutrAvidin (Cat. no. 31050, ThermoFisher Scientific) at RT for 24 hours. Crosslinked complex of unconjugated goat anti-rabbit antibodies (Cat. no. A16112, Invitrogen) and AlexaFluor 594-conjugated donkey anti-goat antibody Fab fragment (Cat. no. 705-585-003, Jackson ImmunoResearch) were prepared as above as 10 μl reaction mixtures and diluted 1:16000 in PBST. Each well was coated with 100 μl of NeutrAvidin solution at 1:100 dilution overnight at 4° C. The NeutrAvidin-coated wells were washed with PBST for 5 minutes×4 times at RT, and then aspirated clean. The wells were then blocked with 5% w/v BSA at RT for 2 hours, then washed with PBST for 5 minutes×4 times. The wells were then coated with 100 μl of the diluted crosslinked antibody-Fab complex reaction mixture (diluted to 1:16000) at RT for 2 hours. The wells were then aspirated, washed 5 minutes×4 times at RT with PBST, incubated with 100 μl of 0.01 mg/ml rabbit IgG isotype (Cat. no. 02-6102, ThermoFisher Scientific) at RT for 2 hour. After aspiration and washing with PBST for 5 minutes×4 times, 100 μl of HRP-conjugated goat anti-rabbit antibodies (diluted to 0.5 μg/ml with 1× PBS, Cat. no. P0448, Dako, Santa Clara, Calif.) were added and incubated at RT for 2 hour. After aspiration and washings, 100 μl of freshly made substrate solution of 2,2′-azinobis[3-ethylbenzothiazoline-6-sulfonic acid]-diammonium salt (AB TS; Cat. no. 10102946001, Sigma-Aldrich) at 5.71 mM with 0.03% w/w H₂O₂ in 1× PBS was added to the wells and incubated for 20 minutes at RT. The colorimetric readout was performed on a Victor3 spectrophotometer (PerkinElmer, Waltham, Mass.) with 0.1 second exposure at 405 nm. Data and statistical analyses were performed using the Prism software (v8, GraphPad, San Diego, Calif.).

Functional Assessment of SPEAR Heat Resistance with a Hot-Start PCR Assay

Mouse anti-Taq antibodies (Genscript A01849, Piscataway, N.J.) were made into Taq SPEARs as described above with (conventional SPEARs) or with 61.8 mM pyridine (SPEARs^{py}). The crosslinking duration was 4 hours at 13° C. for both groups. The reaction products were purified using Amicon ultracentrifugal filters with MWCO of 30 kDa (UFC503096, Millipore, Burlington, Mass.) and diluted to 1 unit Taq SPEAR per 10 μl, 1× PBS. The purified Taq SPEARs^{(py)} were then split into two groups, one heated at 55° C. for 16 hours and another stored at 4° C. until use. To setup the hot-start PCR functional assessment assay, 1 unit anti-Taq antibody or Taq SPEARs^{(py)} (heated or non-heated) was mixed with 0.1 μM forward and reverse primers (GCGTGCACTTTTTAAGGGAGG and CAGTATTTTTCCGGTTGTAGCCC, respectively), 0.1 ng template (plasmid #25361, Addgene) and PCR master mix (TaKaRa R004A, Shiga, Japan) as 30 μl reactions. The PCR thermocycling protocol was as follows: 55° C. for 30 seconds, 25× cycles of 55° C. for 1 minute and 37° C. for 10 minutes, 3× cycles of 95° C. for 1 minute and 60° C. for 1 minute and 72° C. for 1 minute, 72° C. for 10 minutes, and 4° C. infinity hold. The PCR products were then analyzed on 1% agarose gel and imaged using a BioRad Gel Doc EZ System with automatic exposure. The obtained gel band intensities were measured using Fiji with manually defined ROIs, the quantification procedures have been kept constant for all bands within the same set of experiments. Data and statistical analyses were performed using the Prism software (v8, GraphPad).

### EXAMPLES

All patents, patent applications, provisional applications, and publications referred to or cited herein are incorporated by reference in their entirety, including all figures and tables, to the extent they are not inconsistent with the explicit teachings of this specification.

The following are examples that illustrate procedures for practicing the invention. These examples should not be construed as limiting. All percentages are by weight and all solvent mixture proportions are by volume unless otherwise noted.

### Example 1—Creating Thermostable Antibodies for Immunostaining

Operationally, this method for creating thermostable antibodies involves a 10-minute room temperature incubation of antigen-binding fragments of immunoglobulins, preferably Fab fragments of secondary antibodies or V_{HH} domain fragments of secondary antibodies, with its target primary antibody. The antigen-binding fragments of immunoglobulins are incubated with the primary antibody of interest at about a 1:1 to about a 3:1 molar ratio for 10 minutes at room temperature, with the primary antibody at a final concentration of about 0.1 to about 1 mg/ml. Then, 1× phosphate-buffered saline or 0.1M sodium carbonate buffer is added.

To prepare the cross-linkers, the homo-multifunctional cross-linker, Polyglycerol polyglycidyl ether (P3PE), is diluted into about a 10% to about a 20% v/v solution in water, vortexed for 1 minute at room temperature to ensure emulsion formation, and then centrifuged to obtain the clear supernatant solution. The supernatant is then added to the primary antibody, antigen-binding fragments of immunoglobulins, and buffer mixture at about a 1:5 dilution. The cross-linkage will then be allowed to proceed for 24 hours before quenched with the quenching reagent 1M ammonium chloride in 1× PBS or 1M lysine in 1× PBS. The quenched mixture can then be directly used for immunostaining.

### Example 2—Immunolabeling Biological Tissues

To immunolabel tissues, the thermostable antibody mixture is added to the tissue with 4% SDS, 10% sodium deoxycholate, or 0.3% Triton X-100, dissolved in 1× PBS. The mixture is heated to 55° C. for 1 to 10 hours and cooled to room temperature for 1 hour. The immunolabeling can be visualized using the Fab second antibodies that are Alexa Fluor® 594-labeled Fab fragment of secondary antibody. The Alexa Fluor® 594-labeled Fab fragment of secondary antibodies absorb light around 591 nm and fluoresce with a peak around 614 nm.

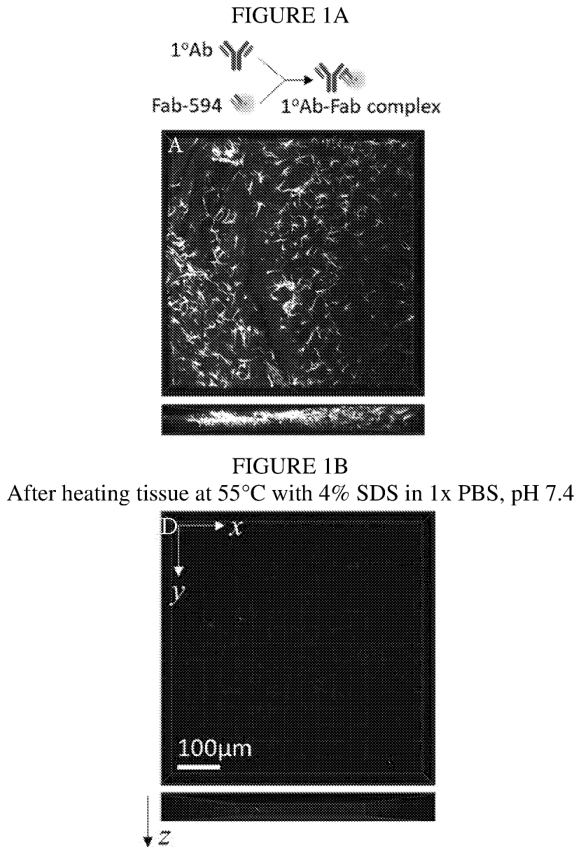

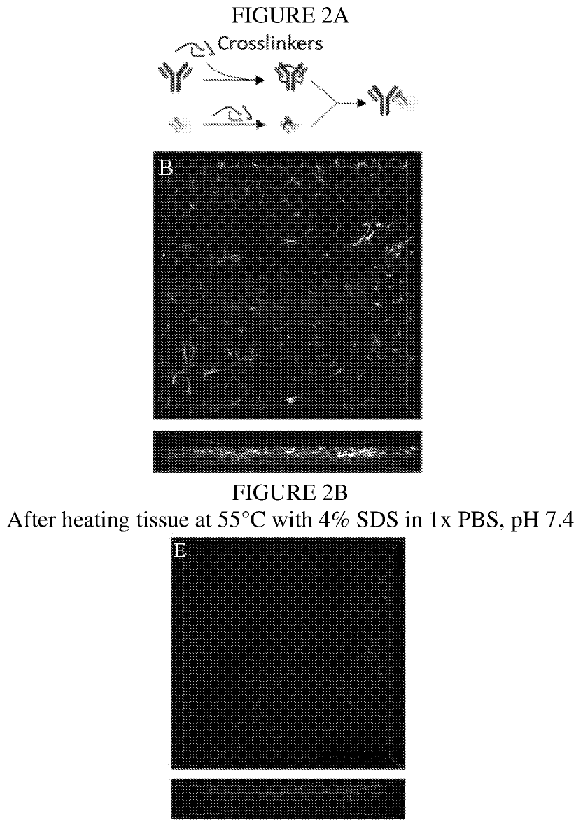

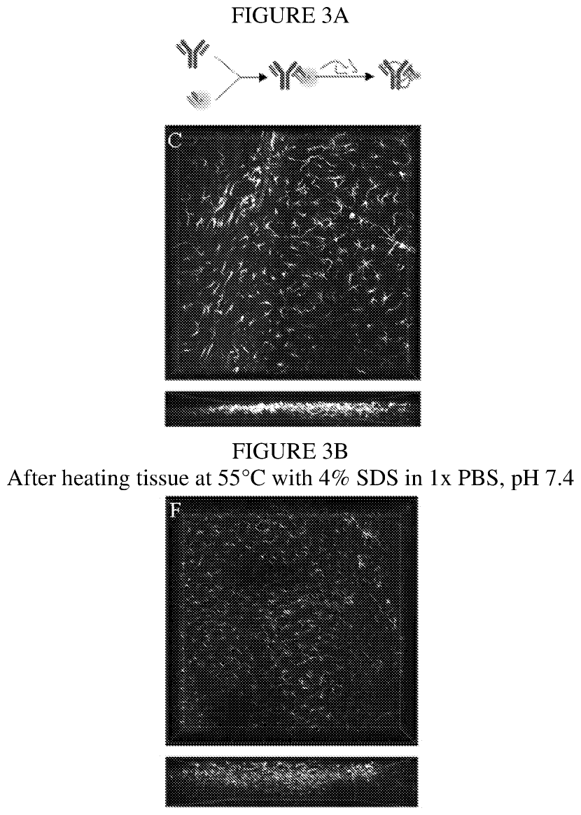

As shown in the FIGS. 1A-1B, 2A-2B, and 3A-3B, there is a clear visualization improvement with the combination of the antigen-binding fragments of immunoglobulins and its target primary antibody with a buffer followed by the addition of the cross-linkers. The added components in the order demonstrated in FIGS. 3A and 3B provide an increased staining depth as shown in FIG. 3B. In either of the cases in which the cross-linkers were added before an incubation of the antigen-binding fragments of immunoglobulins and its target primary antibody, there is minimal immunolabeling, particularly at depth.

### Example 3—Thermo-Immunohistochemistry with Optimized Kinetics (ThICK) Staining Protocol

Fresh brain tissues were obtained and stored as described above. Tissues <300 μm-thick were permeabilized for 1 day in PBST at 37° C., while larger samples were treated with 4% w/v SDS in 0.2 M borate buffer, pH 8.5 at 37° C. until optically transparent. The permeabilized sample was then washed thoroughly in PBST at 37° C. for 3 times (1 hour each). This is essential as any residual SDS will precipitate with GnCl used in the next step. The washed sample was then equilibrated in roughly five-times the tissue volume of PBST with 1 M GnCl at 55° C. for 30 minutes, after which 10 μl of SPEAR reaction mixture per 100 μl staining buffer was added to the staining solution and incubated at 55° C. for 16-72 hours, depending on the sample thickness. The staining duration can be increased by 8 hours for every 200 μm staining depth, although it is likely that optimization of antibody concentration and staining duration will be required for individual antibody-antigen pairs. After incubating at 55° C., the sample was cooled to room temperature and incubated further for 1 hour. The sample was then briefly washed in PBST to remove any residual GnCl and incubated in OPTIClear2 for 2 hours or OPTIClear for 6 hours at 37° C. The optically cleared sample can then be imaged.

### Example 4—Large Scale Human Pons Section Thick-Staining with SPEAR^{py}

A human postmortem brainstem sample was fixed in 10% neutral buffered formalin for 3 weeks before washing and storage in PBS at 4° C. A 5 mm-thick transverse section of the pons was then cut. The pons slice was then sectioned sagittally and cut posterior to the medial lemniscus to obtain a subdivision containing the locus coeruleus. The sample was then permeabilized in 4% w/v SDS in 0.2 M borate buffer, pH 8.5 at 55° C. for 24 hours and washed three times in PBST, 2 hours each. Meanwhile, TH SPEAR^{py} (with AlexaFluor 594) was prepared from 30 μl rabbit anti-TH antibody (AB152, Millipore) with 16 hours of incubation at 13° C. for 24 hours. The washed sample was then placed in 3 ml fresh PBST with 300 μl TH SPEARs^{py} reaction mixture and ThICK-stained at 55° C. for 24 hours. After ThICK staining, the sample was cooled to 4° C. overnight, washed in PBST briefly for 1 hour at RT, and incubated in 20 ml OPTIClear at 37° C. overnight.

The stained and cleared sample was then imaged using an in-house custom built two-photon microscope in tiled Z-stack mode (total acquisition field-of-view of 1773×2754×1084 μm³) using an Olympus XLPLN10XSVMP (10×, NA 0.6, WD 8 mm) objective. The Z-stacks were then imported into Zen Blue software (ZEN 3.3, Carl Zeiss, Oberkochen, Germany). Gaussian blurred Z-stacks were then generated from each tile and used to correct shading inhomogeneity. The adjusted images were then background subtracted. Stitching was performed using the ImageJ plugin BigStitcher. The stitched image was imported into Imaris (v9, Bitplane) and cells were segmented with local background contrast option and filtered based on volume and sphericity parameters, followed by manual refinements. To quantify cell distance from the nearest tissue surface, a surface was generated encompassing all voxels outside of the tissue. A new channel with a linear gradient of voxel intensity that scales with the distance from the above generated surface was created using distance transformation in MATLAB (R2018b, MathWorks). The mean intensities of the distance transformation channel for the segmented cell surfaces were thus their distance from the nearest tissue surface.

For comparison, a 1.5 mm-thick human pons samples that also contained the locus coeruleus was fixed in 10% neutral-buffered formalin for 3 weeks, permeabilized in 4% w/v SDS in 0.2 M borate buffer, pH 8.5 at 55° C. for 24 hours and washed three times in PBST, 2 hours each. 10 μl of Rabbit anti-TH antibody was then added every day to the immunostaining PBST solution to a total of 100 μl, and the tissue was then incubated for an additional 4 days at 37° C. The sample was then washed in PBST overnight for 1 day, and AlexaFluor 594-labeled donkey anti-rabbit secondary antibody (Invitrogen, R37119)) was applied in a similar regimen. The sample was then washed and cleared in OPTIClear overnight. Imaging was performed with a Carl Zeiss LSM 780 confocal microscope using a 10× objective (Carl Zeiss Plan-Apochromat 10×/NA 0.45 M27) with an imaging depth of 1,500 μm (i.e. full-thickness imaging). Stitched was performed alongside acquisition in Zen Black software (ZEN 2.3, Carl Zeiss). Subsequent image analyses and cell segmentation was identical to the TH SPEAR^{py} labeled sample as described above.

### Example 5—Cross-Linking IgG To Fab

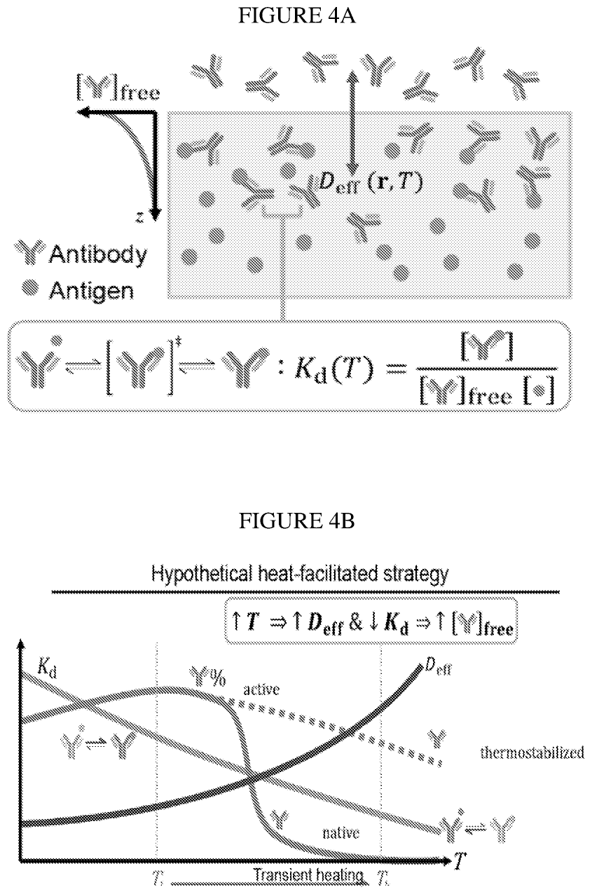

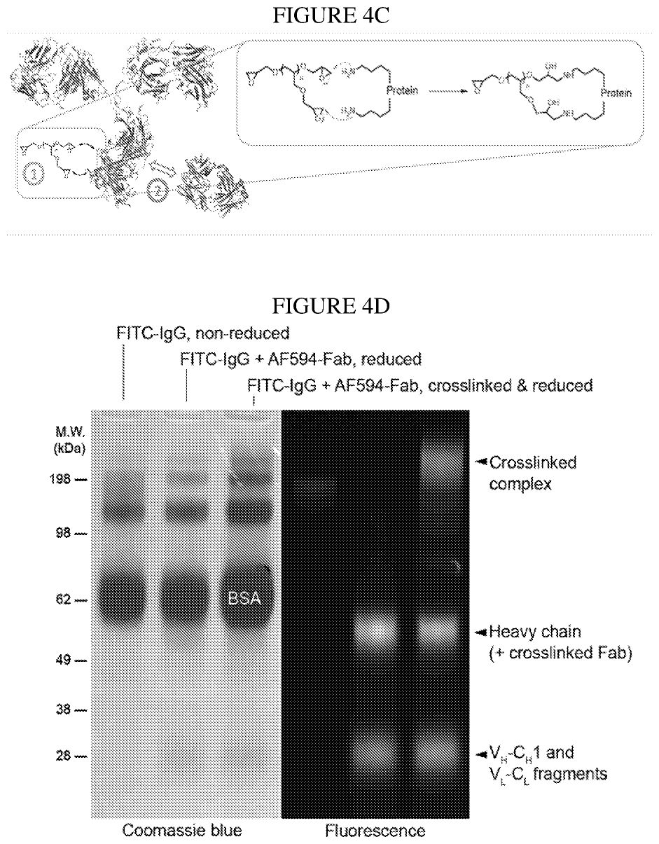

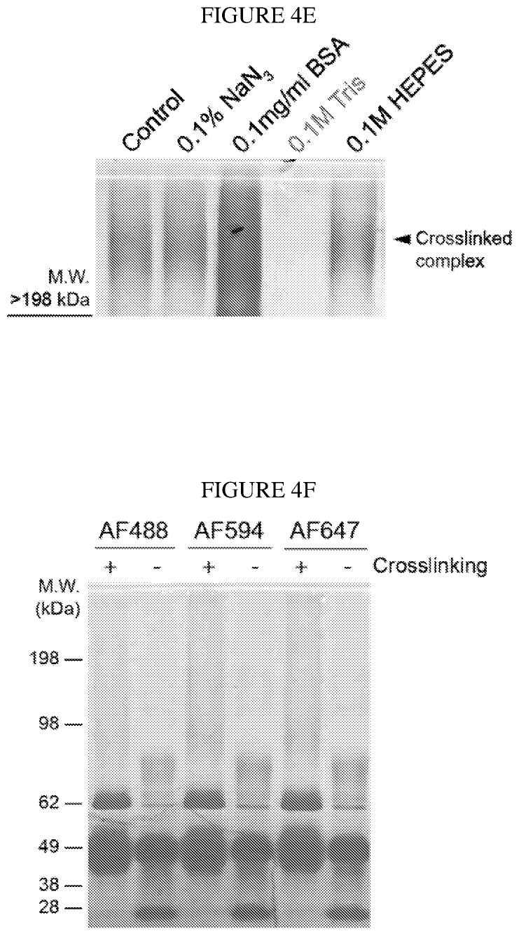

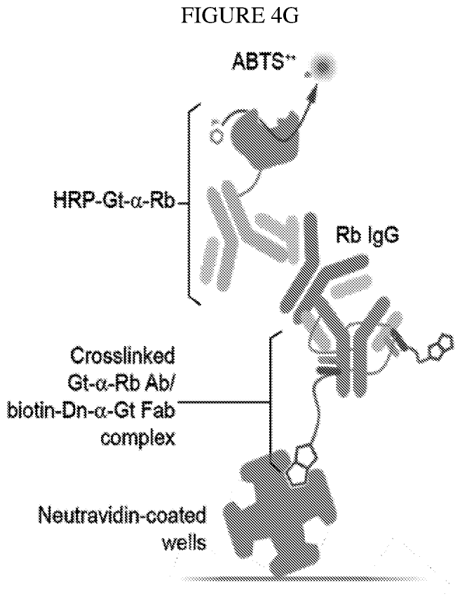

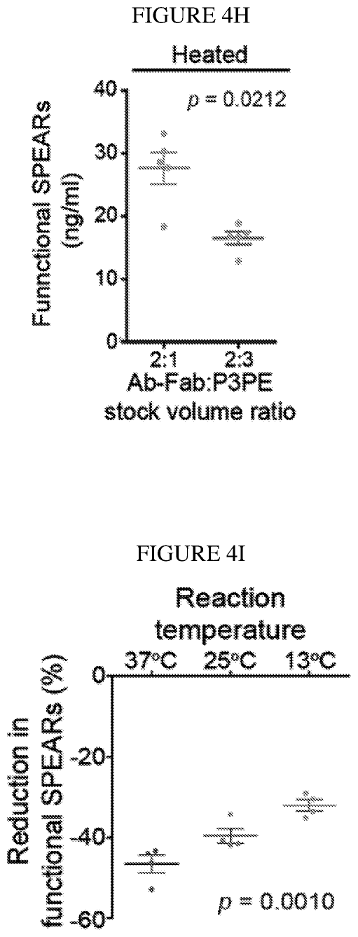

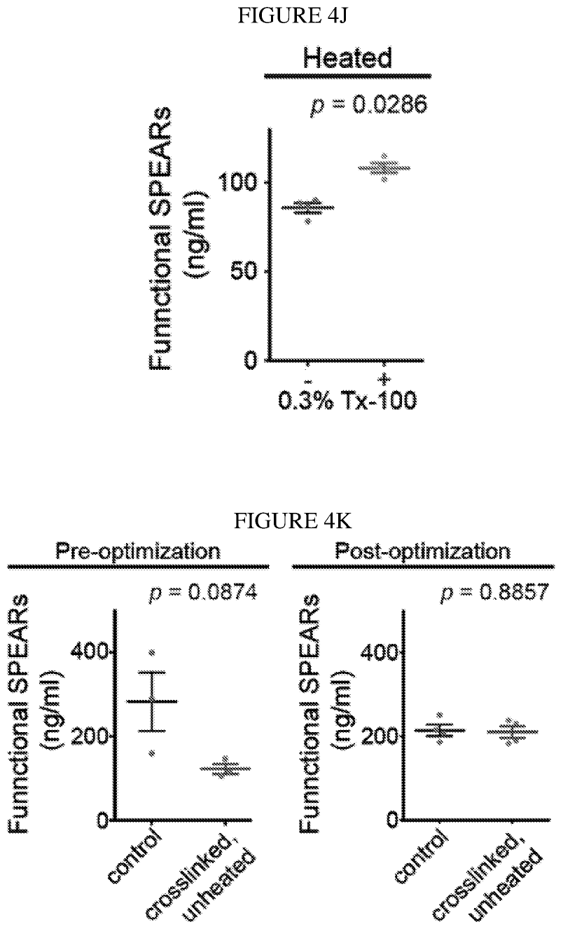

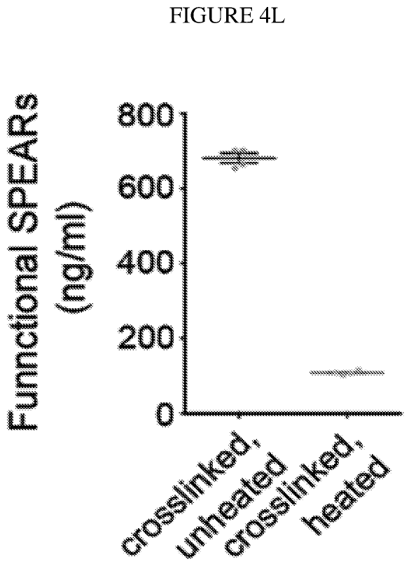

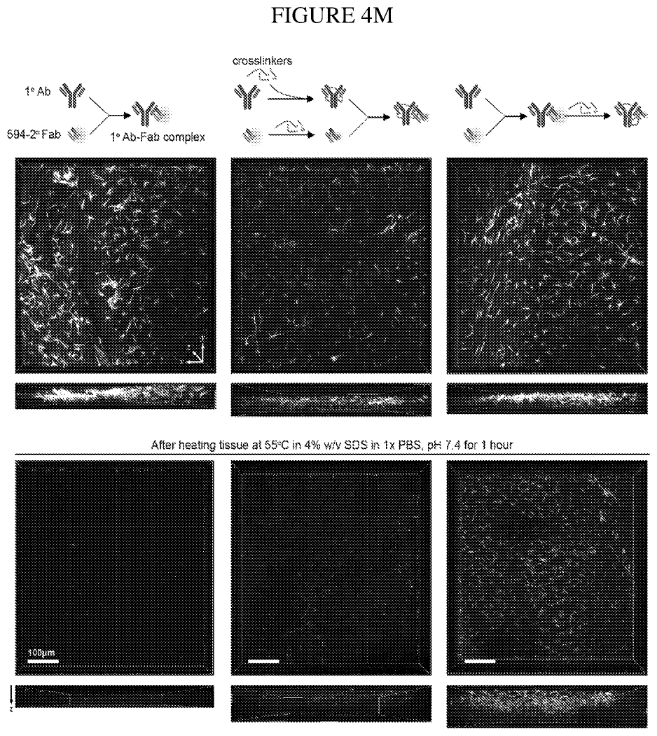

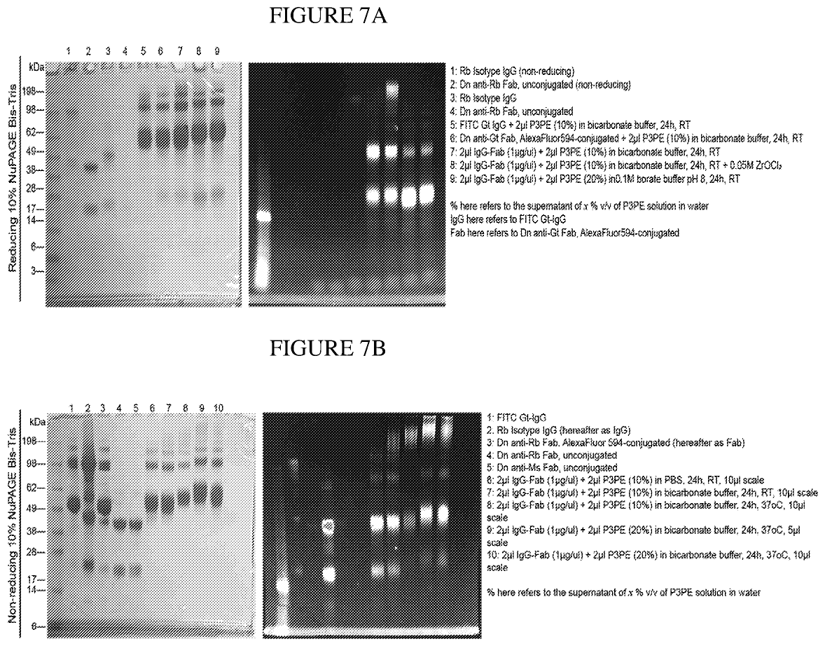

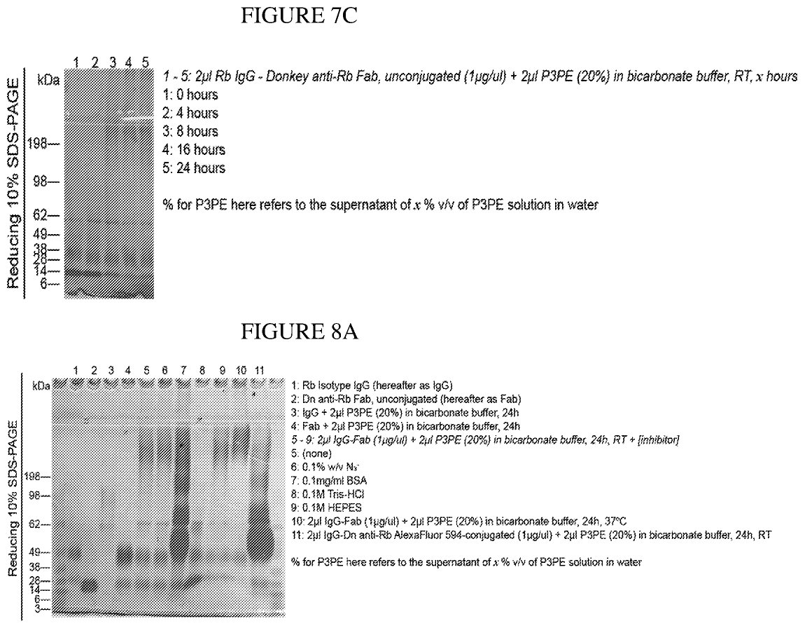

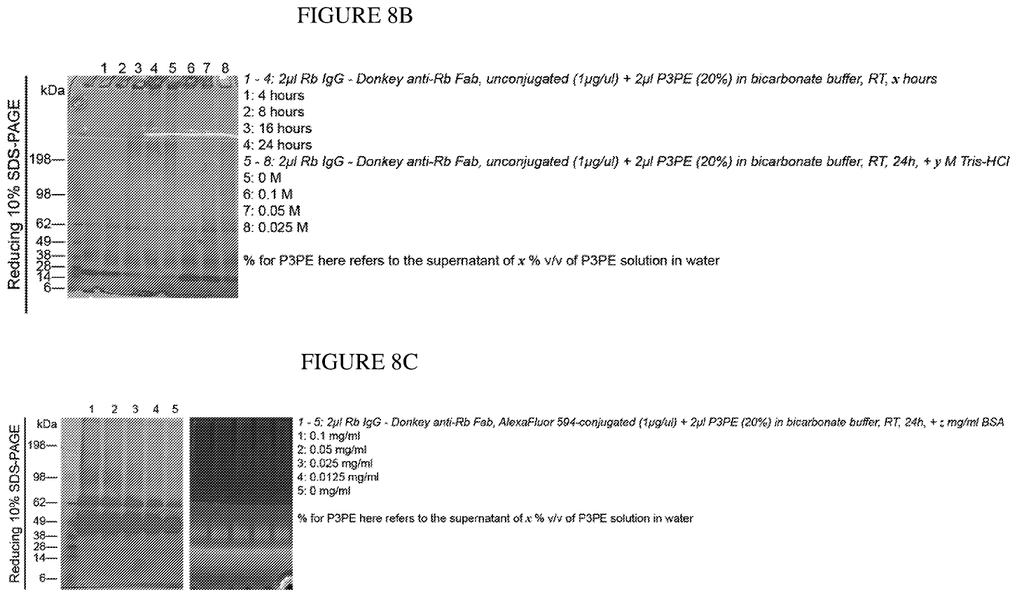

Using fluorescently labeled Fab fragments of secondary antibodies (hereafter referred to as Fab), we first identified and optimized the reaction condition that leads to the reliable formation of a crosslinked immunoglobulin G (IgG)-Fab complex in a reasonable time (<24 hours) and reaction scale (10 μl reaction per 0.1-1 μg antibody). The crosslinking can be unambiguously confirmed using reducing SDS-PAGE with fluorescent readout of AlexaFluor 594-labeled Fabs (FIG. 4D, FIGS. 7A-7C). We tested and confirmed that the P3PE crosslinking reaction is compatible with most additives, buffer components, and preservatives in commercially available antibody liquors, except Tris base due to its primary amine group (FIG. 4E, FIGS. 8A-8C). The choice of conjugated fluorophore on Fabs does not affect the efficiency of the P3PE crosslinking reaction (FIG. 4F).

### Example 6—Assessing Functionality and Optimization of the Antigen-Binding Capability and Heat Stability of SPEARs

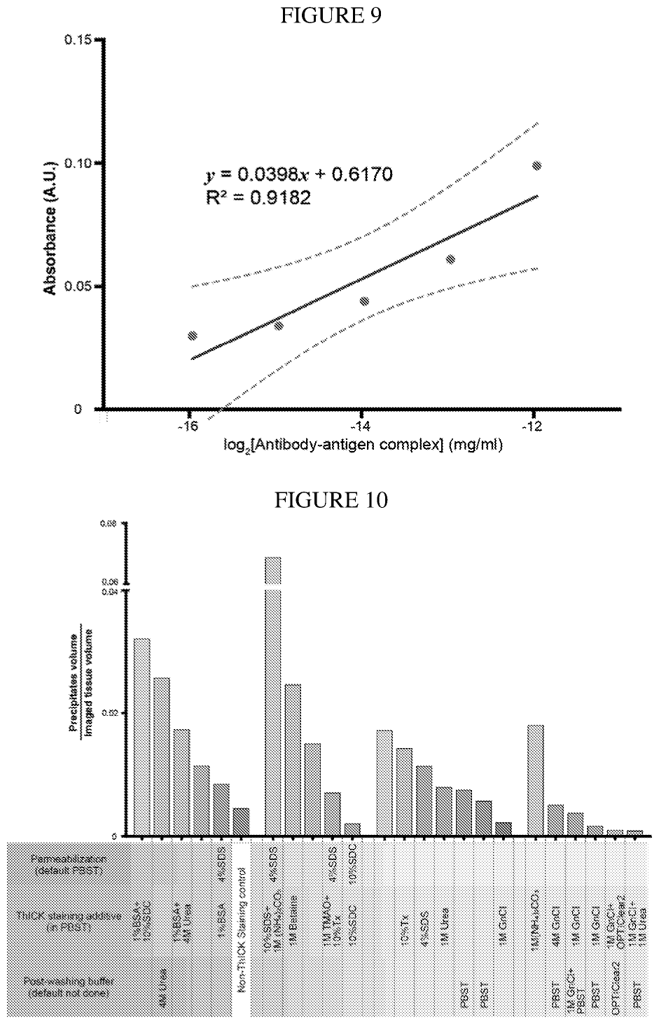

We next designed and utilized an enzyme-linked immunosorbent assay (ELISA) variant that can functionally assess and optimize the antigen-binding capability and heat stability of the SPEARs in a high-throughput manner (FIG. 4G, FIG. 9). We found that a higher Ab-Fab complex:P3PE molar ratio (FIG. 4H), a lower temperature of crosslinking (FIG. 1I), an optimal duration (16-24 hours) of crosslinking, and heating in the presence of 0.3% w/v Triton X-100 (FIG. 4J) resulted in better antigen-binding capability and thermostability of the so-formed SPEARs. After optimization, the antigen-binding capability of the SPEARs improved from 43% to 98% of the uncrosslinked control after optimization (FIG. 4K), and 15.9% crosslinked SPEARs still remained functional after heating at 55° C. for 16 hours (FIG. 4L). In a proof-of-concept immunostaining test using rat anti-GFAP antibody and AlexaFluor 488-labeled donkey anti-rat antibody Fab fragments, we found that P3PE crosslinking and Fab-complexation can synergize in stabilizing antibodies against heat and denaturant destruction (FIG. 4M).

### Example 7—Using SPEARs in ThICK Staining

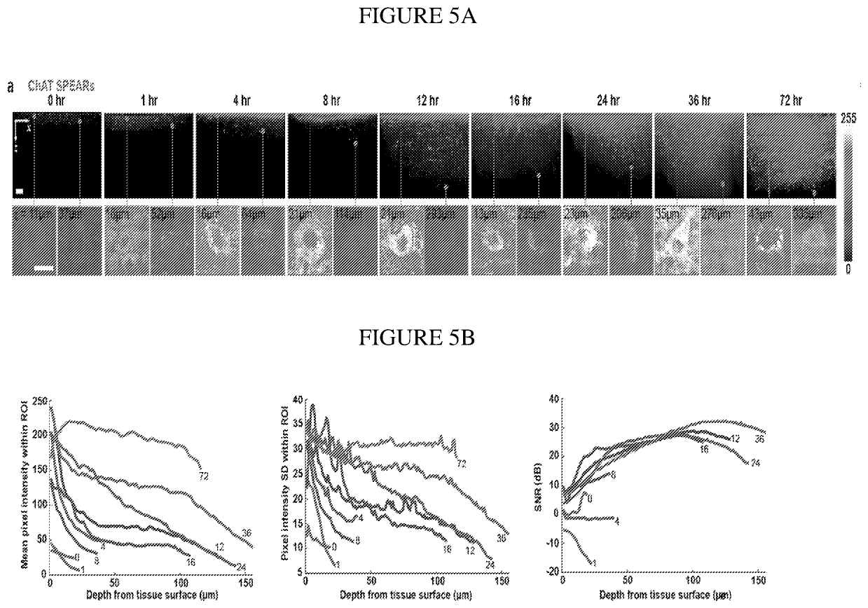

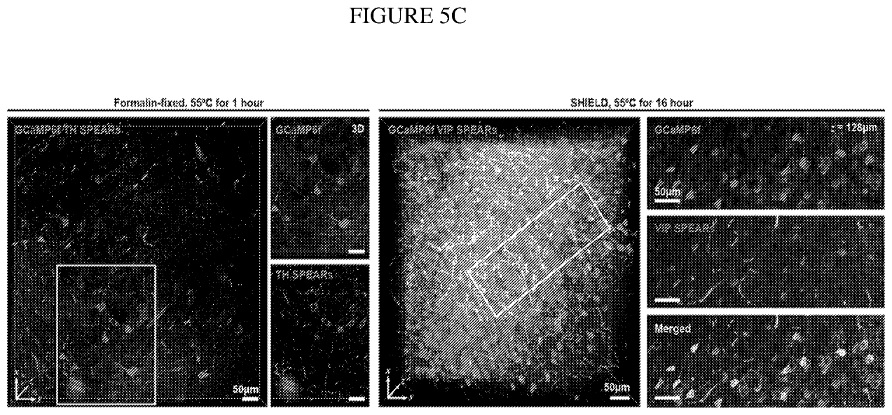

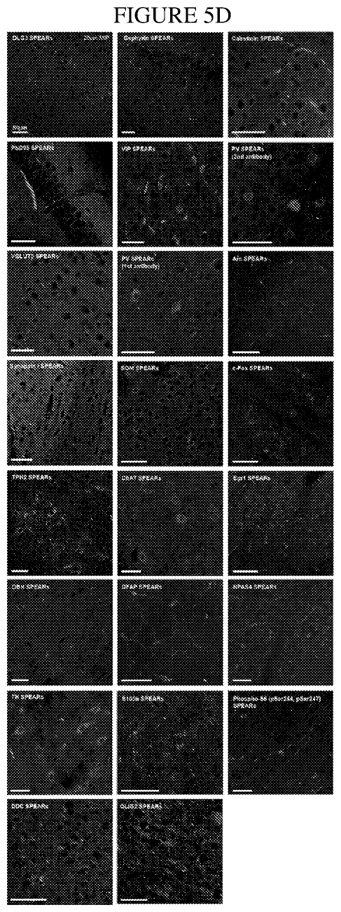

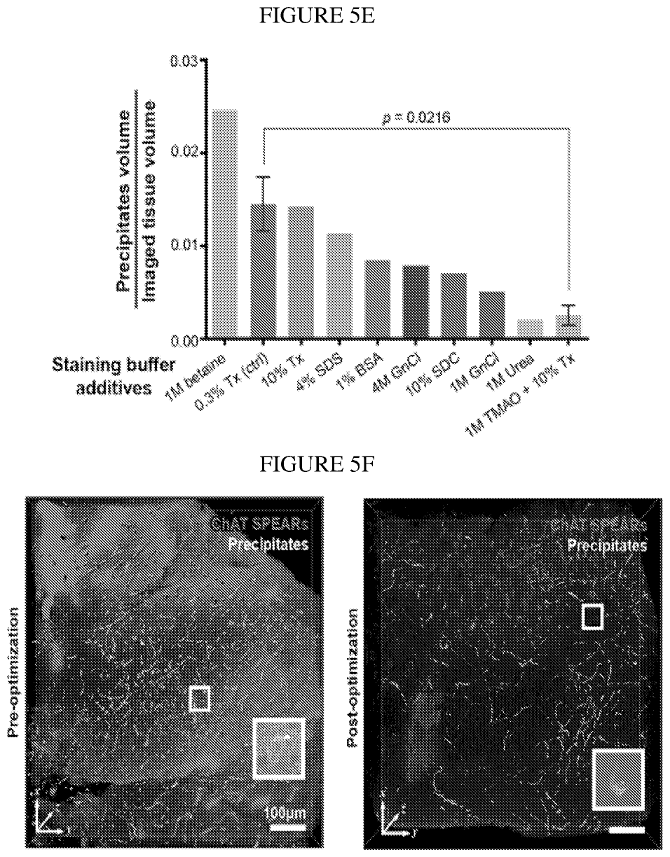

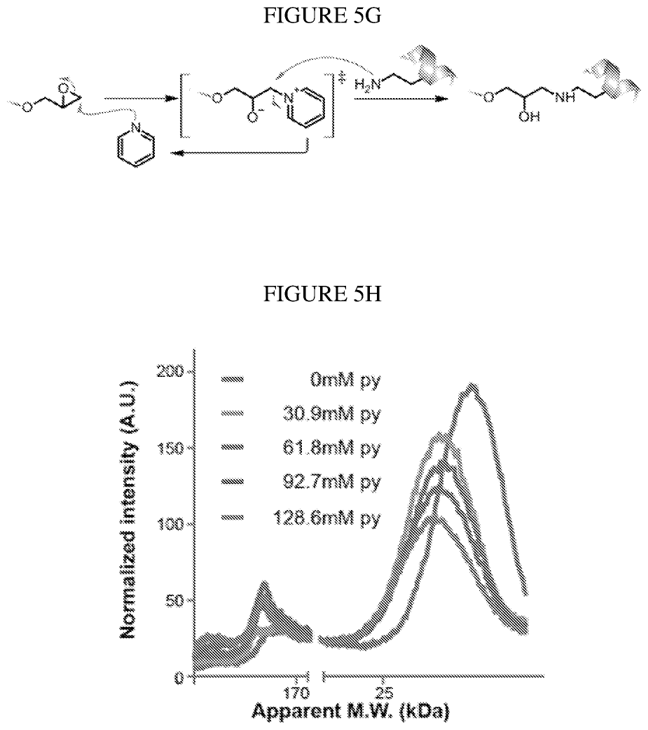

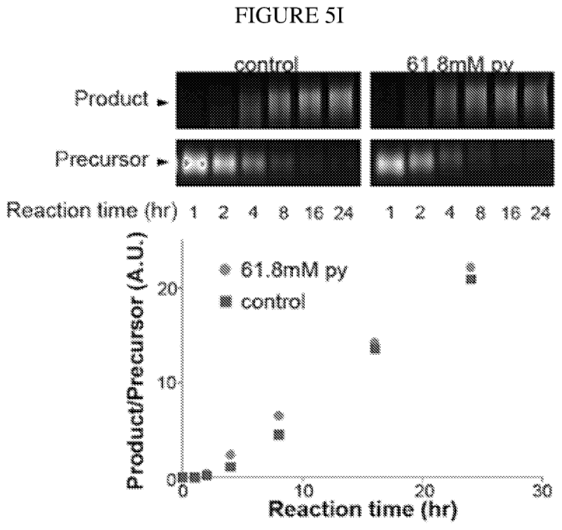

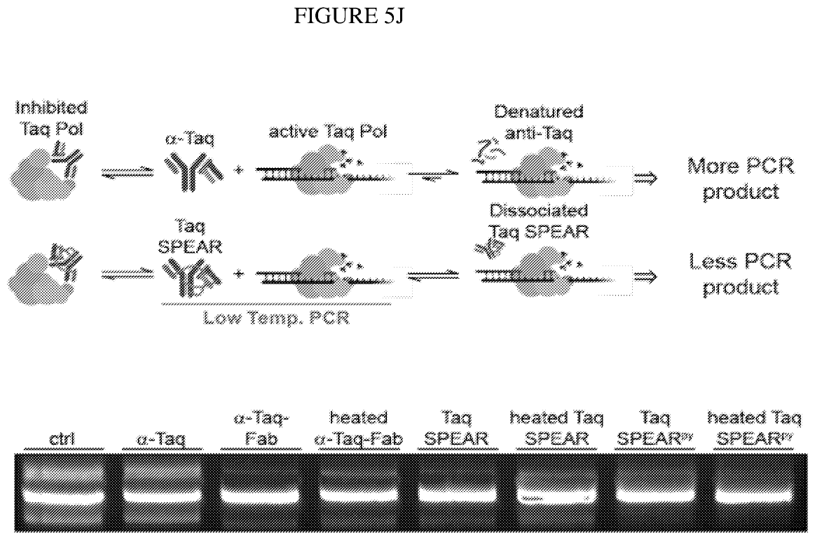

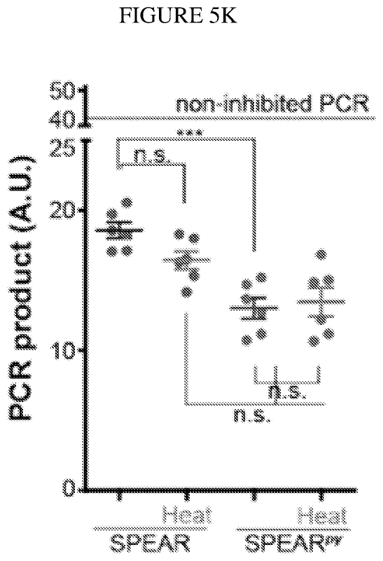

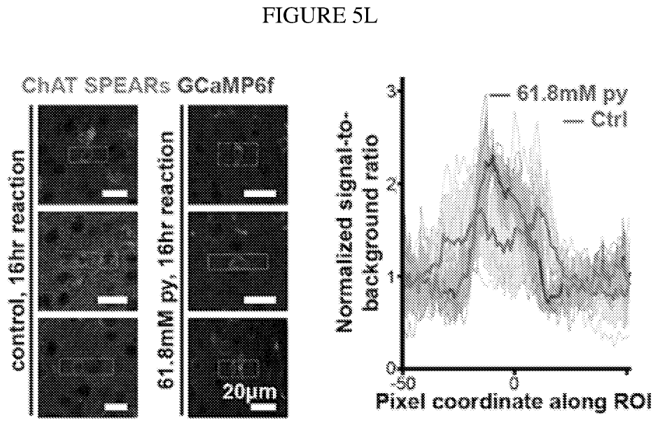

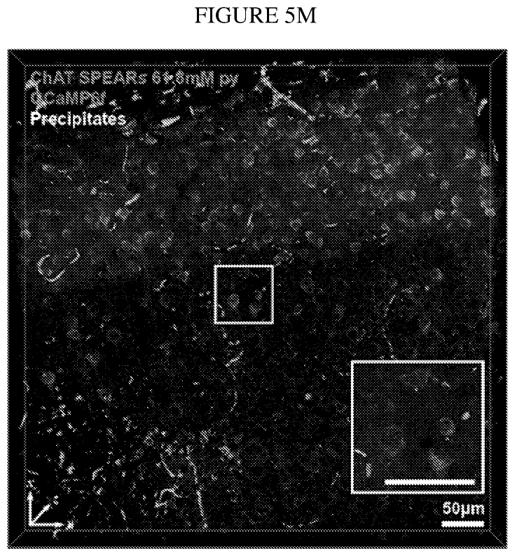

After obtaining optimally heat-resistant SPEARs, we next determined their applicability to ThICK staining. We found that SPEARs can tolerate heating at 55° C. in PBST for at least 72 hours (FIG. 5A) and observed that the signal homogeneity across tissue depth positively correlated with the duration of heating (FIG. 5B). For samples with endogenous fluorescence preserved by conventional formalin fixation, the thermal sensitivity of fluorescent proteins limits the duration of heating to 1 hour at 55° C. in PBST (FIG. 5C). Nonetheless, the application of SHIELD protection allowed heating to be extended to at least 16 hours (FIG. 5C). Testing the scope of applicability, in addition to the anti-GFAP antibody above, we tested and verified that SPEARs can be readily produced from 23 other commercially available primary antibodies (including various neuronal subtype, activity, synaptic and glial markers, see Table 1) for ThICK staining at 55° C. in PBS with 0.3% Triton X-100 (PBST) for 16 hours (FIG. 5D).

TABLE 1

| | | | Antibody | |
|---|---|---|---|---|
| Antibody | | | concentration | |
| target | Host | Supplier/Cat. no. | (mg/ml) | Additives |
| Arc | Ms | Santa cruz | 0.2 | 0.1% NaN₃,, 0.1% gelatin, |
| | | biotechnology | | 1× PBS |
| | | sc-17839 | | |
| CR | Rb | Abcam ab702 | 5.860 | 0.09% NaN₃, “Carrier protein”, |
| | | | | 1× PBS, pH 7.3, Van Gogh |
| | | | | yellow diluent |
| ChAT | Gt | Millipore AB144P | 3.006 | In buffer with 5 mg/ml BSA, |
| | | | | 0.2% NaN3 |
| DBH | Rb | Sigma HPA002130 | 0.1 | 40% glycerol, 0.02% NaN₃, |
| | | | | 1× PBS |
| DDC | Rb | Sigma HPA017742 | 0.05 | 40% glycerol, 0.02% NaN₃, |
| | | | | 1× PBS |
| DLG3 | Rb | Sigma HPA001733 | 0.1 | 40% glycerol, 0.02% NaN₃, |
| | | | | 1× PBS |
| EGR1 | Ms | Santa cruz | 0.2 | 0.1% NaN₃,, 0.1% gelatin, |
| | | biotechnology | | 1× PBS |
| | | sc-515830 | | |
| c-Fos | Ms | Santa cruz | 0.2 | 0.1% NaN₃,, 0.1% gelatin, |
| | | biotechnology | | 1× PBS |
| | | sc-166940 | | |
| Gephyrin | Ms | Santa cruz | 0.2 | 0.1% NaN₃,, 0.1% gelatin, |
| | | biotechnology | | 1× PBS |
| | | sc-25311 | | |
| GFAP | Ms | Santa cruz | 0.2 | 0.1% NaN₃,, 0.1% gelatin, |
| | | biotechnology | | 1× PBS |
| | | sc-58766 | | |
| GFAP | Rt | Invitrogen 13-0030 | 0.351 | 0.1% NaN3, 1× PBS |
| NPAS4 | Rb | Invitrogen PA5- | 1.0 | 50% glycerol, 150 mM NaCl, |
| | | 39300 | | 0.02% NaN₃, 1× PBS |
| OLIG2 | Rb | Sigma HPA003254 | 0.3 | 40% glycerol, 0.02% NaN₃, |
| | | | | 1× PBS |
| Phospho- | Rb | Invitrogen 44-923G | | 50% glycerol, 1 mg/ml BSA, |
| S6 | | | | 0.05% NaN₃, 1× PBS |
| (pSer244, | | | | |
| pSer247) | | | | |
| PSD95 | Ms | NeuroMab K28/43 | 1.0 | 10 mM Tris, 50 mM NaCl, |
| | | | | 0.065% NaN₃, pH 7.4 |
| PV | Rb | Abcam ab11427 | 3.568 (1.0) | 3% BSA, 0.05% NaN₃, 1× PBS |
| PV | Rb | Invitrogen PA1-933 | 1.0 | 20 mg/ml BSA, 0.1% NaN₃, |
| | | | | 1× PBS |
| S100b | Rb | Enzo LifeSciences | 0.2 | 0.1 mg/ml BSA, 0.05% NaN₃, |
| | | ENZ-ABS307-0100 | | 1× PBS |
| Synapsin | Rb | Novus Biologicals | 0.649 (0.1) | 10 mM HEPES, pH 7.5, 0.15M |
| I | | NB300-104 | | NaCl, 0.1 mg/ml BSA, 50% |
| | | | | glycerol |
| SOM | Rt | Millipore MAB354 | (uncertain) | Unpurified tissue culture |
| | | | | supernatant, 0.05% thimerosal |
| TH | Ms | Millipore AB152 | 0.416 | 10 mM HEPES, pH 7.5, 150 mM |
| | | | | NaCl, 0.1 mg/ml BSA, 50% |
| | | | | glycerol |
| TPH2 | Ms | Sigma | 1.0 | 40% glycerol, 0.02% NaN₃, |
| | | AMAb91108 | | 1× PBS |
| VGLUT2 | Ms | Sigma | 0.5 | 40% glycerol, 0.02% NaN₃, |
| | | AMAb91081 | | 1× PBS |
| VIP | Rb | Bioss bs-0077R | 6.472 (1.0) | 1% BSA, 50% glycerol, 0.09% |
| | | | | NaN₃, “aqueous buffer” |

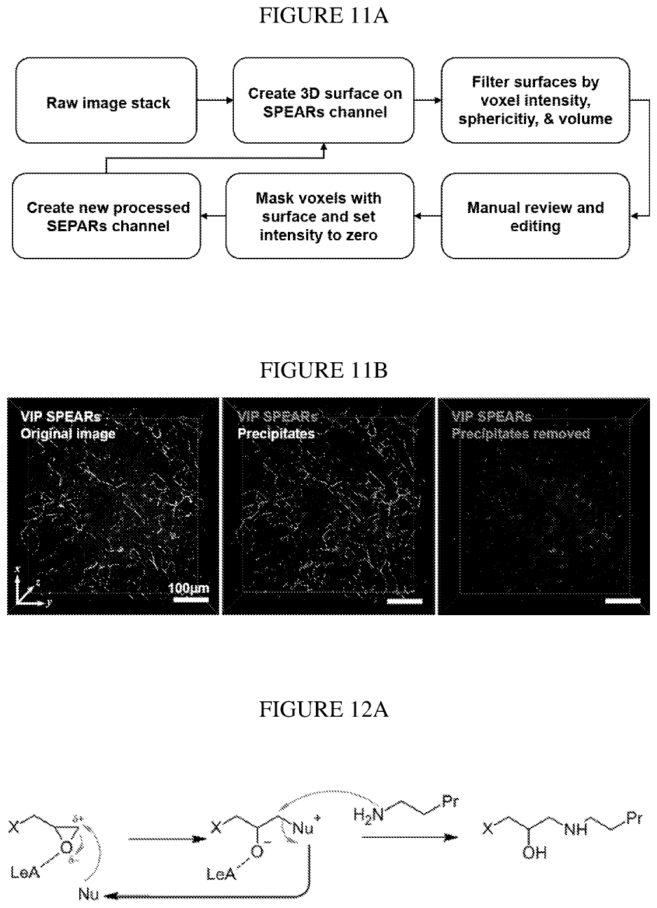

The other challenge in ThICK staining is that SPEARs (and other antibodies in general) commonly precipitate in the vessels, leading to undesired background (FIGS. 5C-5D). The vessels MAY act as low-resistance diffusion channels, where a high protein concentration and inhomogeneous heating lead to denaturation-refolding cycles—a process known to favor the aggregation of antibodies. We thus optimized the staining condition and buffer composition and found that the addition of certain denaturants, notably 1M guanidinium chloride (GnCl) (FIGS. 5E-5F, FIG. 10) can mitigate the precipitation. Alternatively, since the intravascular precipitates typically have high fluorescent intensities and distinct morphology, they can be readily removed by image processing (FIGS. 11A-11B).

### Example 8—Catalyzing Cross-Linking Reactions with Pyridine

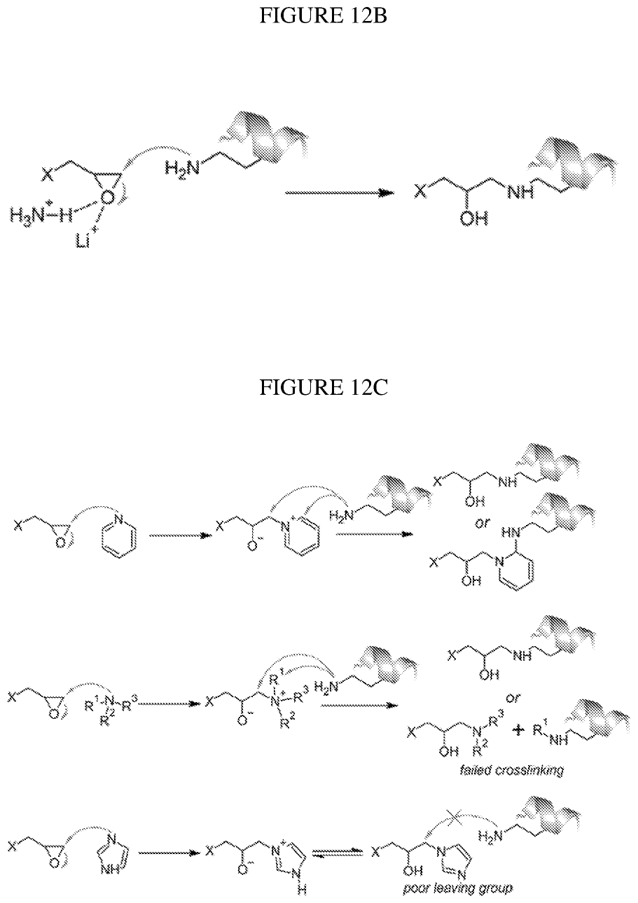

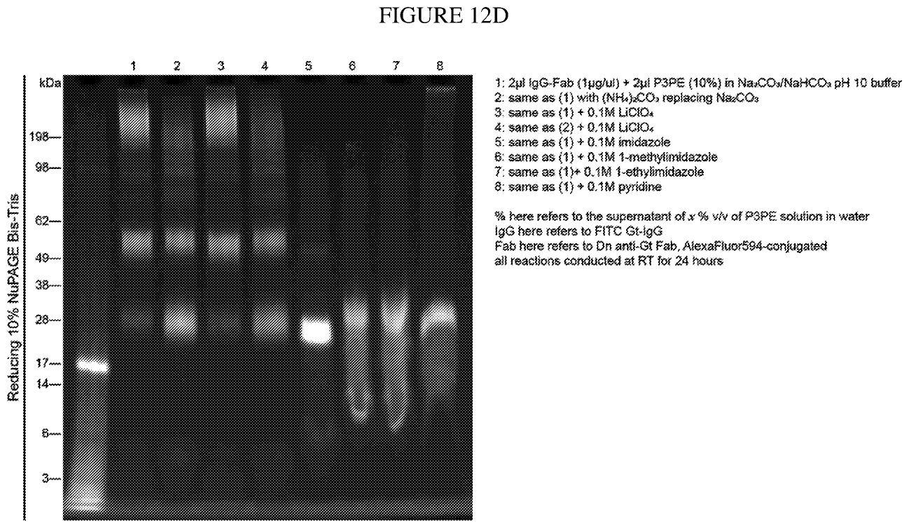

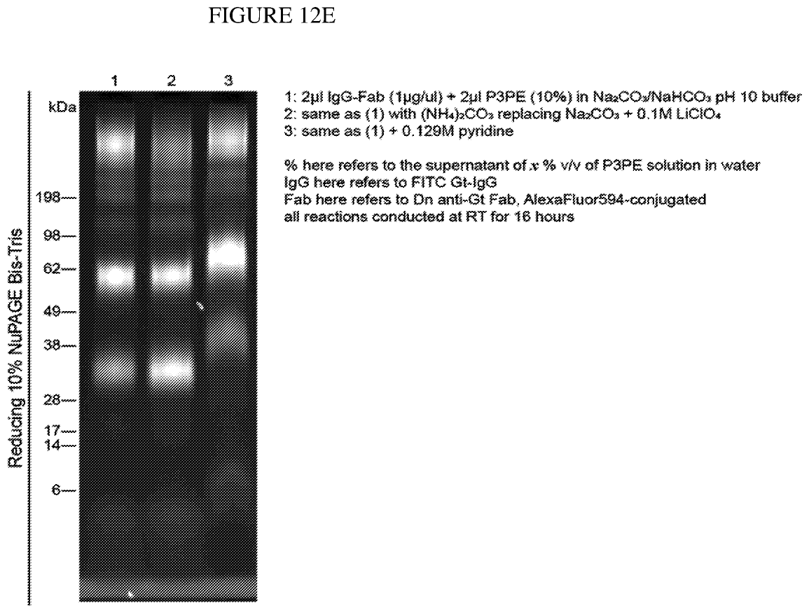

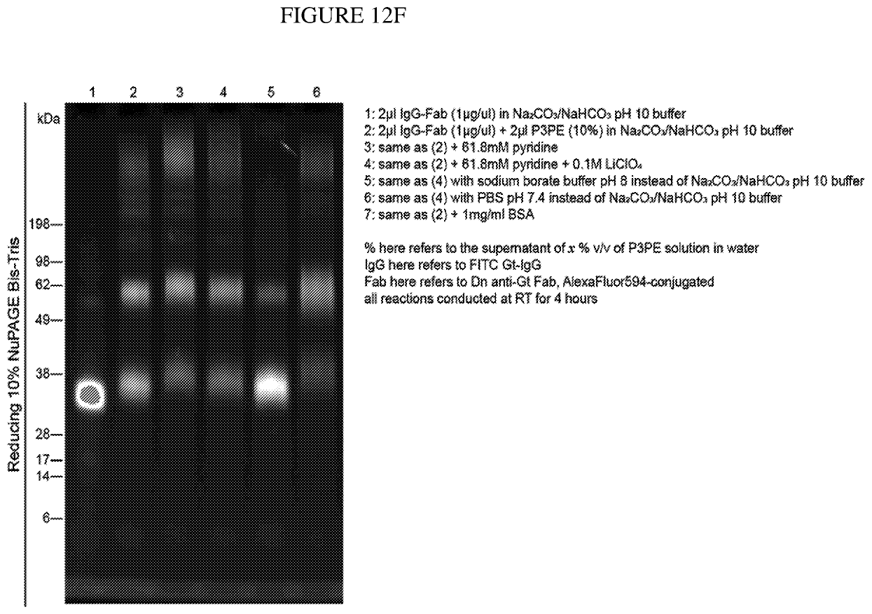

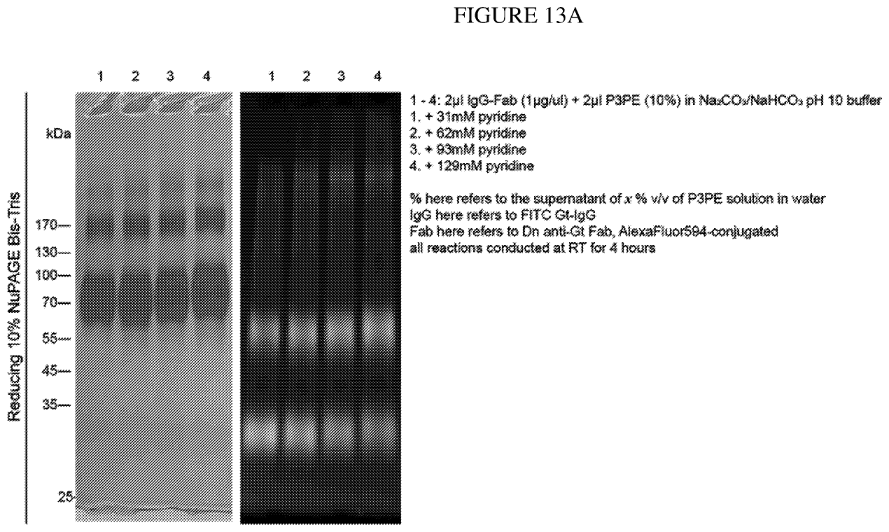

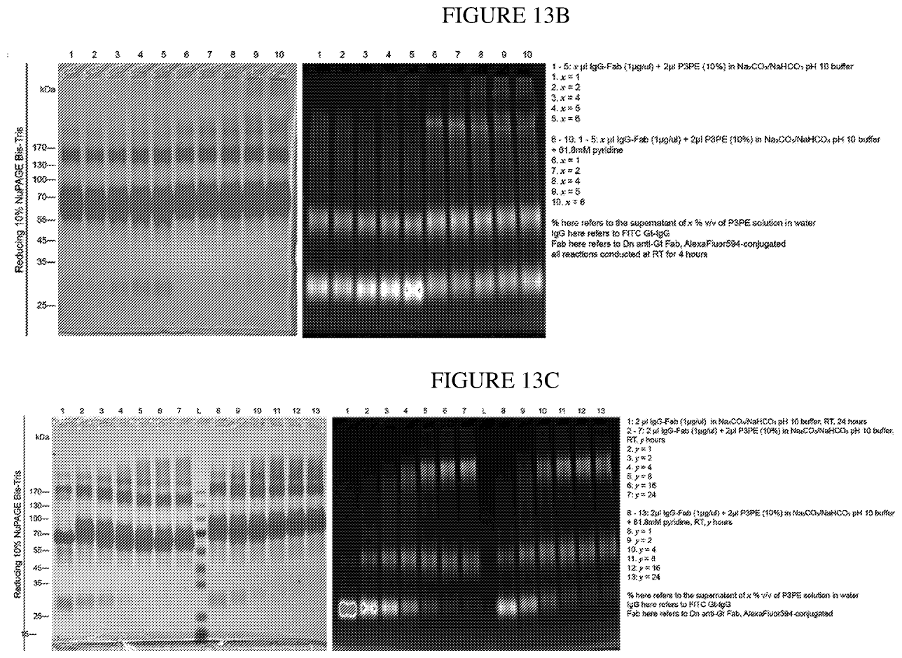

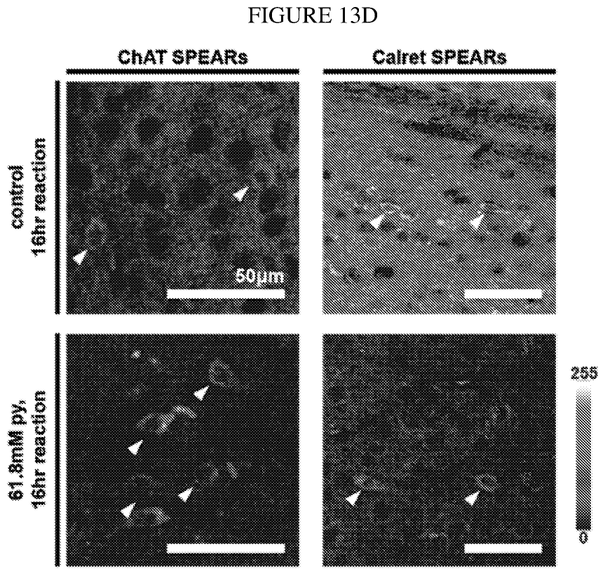

To further streamline the protocol, we explored whether a catalyst can improve the crosslinking reaction speed or yield, we tested pyridine—a moderately strong nucleophile that can form a good pyridinium leaving group when attacked by primary amines (FIG. 5G, FIGS. 12AF, FIGS. 13A-D). We ruled out other catalyst candidates based on theoretical and experimental considerations (FIGS. 12A-12C). Pyridine modestly catalyzed the reaction in a concentration-dependent manner (FIG. 5H) and increased the efficiency of precursor-to-product conversion over 4-8 hours of reaction time (FIG. 5I, FIG. 12AF, FIGS. 13A-D). These catalytically formed SPEARs (denoted as SPEARs^{py}) can be directly used in ThICK staining without additional purification steps, and displayed higher heat resistance in a custom designed hot-start PCR assay (FIGS. 5J-5K). At 16 hours of reaction time, SPEARs^{py} also improved ThICK staining quality compared to that produced by the non-catalyzed reaction (FIG. 5L) and is also compatible with SHIELD-processed samples with endogenous fluorescent proteins (FIG. 5M).

### Example 9—Imaging Human and Mouse Tissue Using Thick Staining with SPEARs

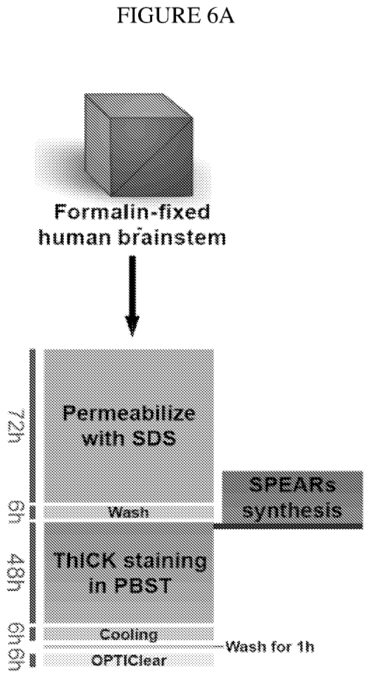

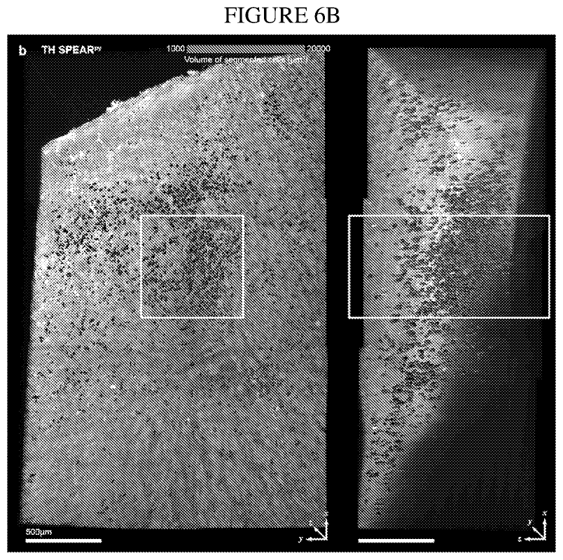

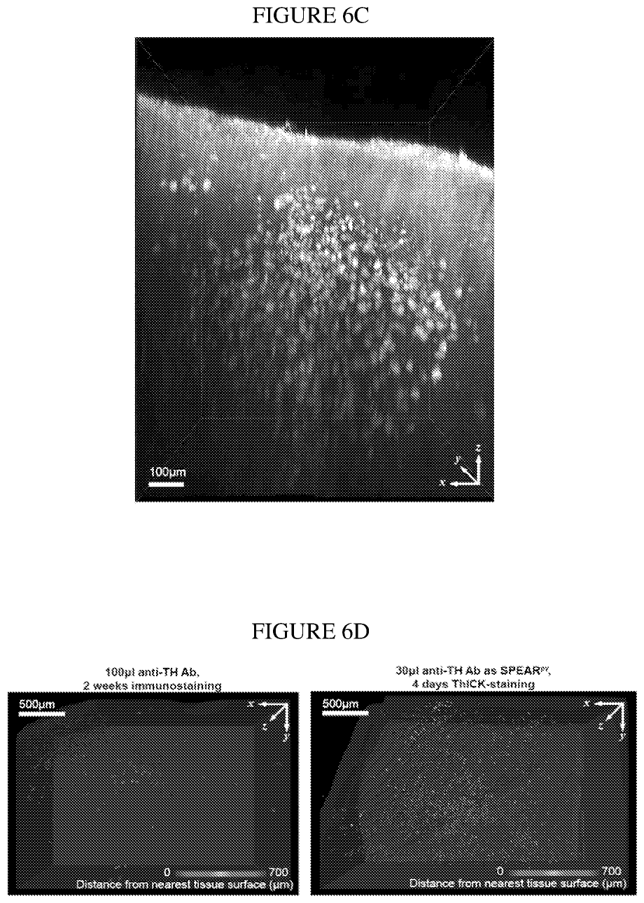

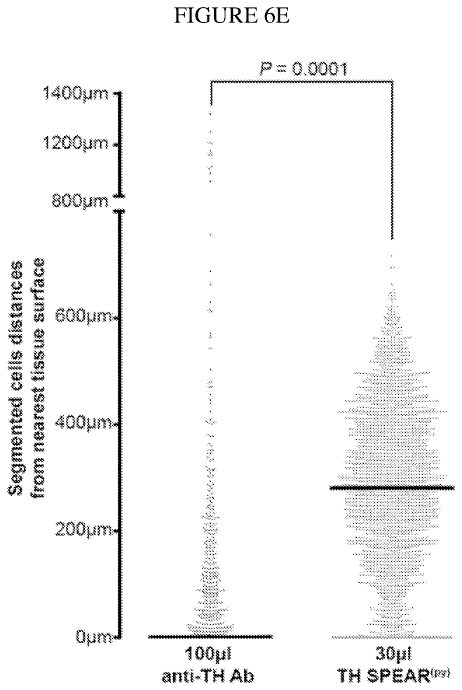

Finally, we applied SPEARs to large-scale three-dimensional imaging of human tissue and the whole mouse brain. We first obtained a 5 mm-thick human pons transverse section inclusive of the locus coeruleus region that has been formalin-fixed for 3 weeks. After 3 days of delipidation and 24 hours of ThICK-staining with 30 μl of TH SPEAR^{py}, we were able to visualize TH-positive noradrenergic cells located as far as ˜700 μm from the tissue surface (FIGS. 6A-6B). In comparison, 2 weeks of conventional immunostaining using 100 μl of TH antibody on a 1.5 mm-thick human pons section from the same region only resulted in ˜80 μm penetration (FIG. 6C). Both the mean penetration depths of segmentable cells and its variance were significantly different (unpaired two-sided t-test, P<0.0001; F-test, P=0.0001, respectively, FIG. 6D).

In conclusion, we have established a fast, user-friendly deep immunostaining method that is readily implementable in most laboratories and compatible with both conventional tissue preservation and tissue clearing methods, especially in conjunction with antigen protection techniques. This is based on a general method for thermostabilizing antibodies (FIG. 4C), which improves their applicability to heat-accelerated deep immunostaining (FIG. 4B) while preserving their antigen-binding property. Producing SPEARs is simple and only requires chemically modifying off-the-shelf antibodies. In principle, SPEARs and ThICK staining can also be applied synergistically with all other existing deep immunolabeling methods, such as those described in Cai, R. et al. Panoptic imaging of transparent mice reveals whole-body neuronal projections and skull-meninges connections. Nat Neurosci 22, 317-327 (2019), Yun, D. H. et al. Ultrafast immunostaining of organ-scale tissues for scalable proteomic phenotyping. Biorxiv 660373 (2019) doi:10.1101/660373, Ku, T. et al. Elasticizing tissues for reversible shape transformation and accelerated molecular labeling. Nat Methods 17, 609-613 (2020), and Susaki, E. A. et al. Versatile whole-organ/body staining and imaging based on electrolyte-gel properties of biological tissues. Nat Commun 11, 1982 (2020), each of which are herein incorporated by reference, to provide the benefits of a more stable antibody and higher macromolecule diffusivity.

### Exemplary Embodiments

Embodiment 1. A method of stabilizing an antibody, comprising combining the antibody with antigen-binding fragments of immunoglobulins to form a mixture, and adding a cross-linker to the mixture.

Embodiment 2. The antibody stabilizing method of Embodiment 1, wherein the antibody is a primary antibody.

Embodiment 3. The antibody stabilizing method of Embodiment 1, wherein the cross-linker is a homo-multifunctional cross-linker.

Embodiment 4. The antibody stabilizing method of Embodiment 3, wherein the homo-multifunctional cross-linker is Polyglycerol-3-polyglycidyl ether (P3PE).

Embodiment 5. The antibody stabilizing method of Embodiment 1, wherein the cross-linker is diluted to about a 1% to about a 50%, or about a 5% to about a 30%, or about a 10% to about a 20% v/v solution in water and then added to the antibody or the antibody and the antigen-binding fragments of immunoglobulins mixture at a dilution of about 1:1 to 1:10, about 1:2 to about 1:8, or about 1:5.

Embodiment 6. The antibody stabilizing method of Embodiment 1, wherein the antigen-binding fragments of immunoglobulins are Fab fragments of secondary antibodies or V_{HH} domain fragments of secondary antibodies.

Embodiment 7. The antibody stabilizing method of Embodiment 1, wherein the antigen-binding fragments of immunoglobulins target immunoglobulins of the primary antibody's host species.

Embodiment 8. The antibody stabilizing method of Embodiment 1, wherein the antigen-binding fragments of immunoglobulins are incubated with the primary antibody at about a 1:1 to about 3:1 molar ratio for 10 minutes at room temperature, with the primary antibody at a final concentration of about 0.1 to 1 mg/ml.

Embodiment 9. The antibody stabilizing method of Embodiment 1, further comprising providing a buffer in the mixture with the primary antibody and antigen-binding fragments of immunoglobulins or in the mixture of primary antibody, antigen-binding fragments of immunoglobulins, and the cross-linker.

Embodiment 10. The antibody stabilizing method of Embodiment 9, wherein the buffer is phosphate-buffered saline (PBS), phosphate-buffered saline and Tween (PBST), or sodium carbonate.

Embodiment 11. The antibody stabilizing method of Embodiment 1, further comprising providing a denaturant in the mixture with the primary antibody and antigen-binding fragments of immunoglobulins or in the mixture with the primary antibody, antigen-binding fragments of immunoglobulins, and the cross-linker.

Embodiment 12. The antibody stabilizing method of Embodiment 11, wherein the denaturant is guanidinium chloride at a concentration of about 0.1 M to about 10 M.

Embodiment 13. The antibody stabilizing method of Embodiment 1, further comprising providing a catalyzing agent in the mixture with the primary antibody and antigen-binding fragments of immunoglobulins or in the mixture of primary antibody, antigen-binding fragments of immunoglobulins, and the cross-linker.

Embodiment 14. The antibody stabilizing method of Embodiment 13, wherein the catalyzing agent is pyridine or a derivative thereof at a concentration of about 1 mM to about 250 mM.

Embodiment 15. An antibody composition comprising a primary antibody, antigen-binding fragments of immunoglobulins, and a cross-linker.

Embodiment 16. The composition of Embodiment 15, wherein the cross-linker is a homo-multifunctional cross-linker.

Embodiment 17. The composition of Embodiment 16, wherein the homo-multifunctional cross-linker is Polyglycerol-3-polyglycidyl ether (P3PE).

Embodiment 18. The composition of Embodiment 15, wherein the cross-linker diluted to about a 1% to about 50%, or about a 5% to about a 30%, or about a 10% to about a 20% v/v solution in water is and the antigen-binding fragments of immunoglobulins at a 1:5 dilution.

Embodiment 19. The composition of Embodiment 15, wherein the antigen-binding fragments of immunoglobulins are Fab fragments of secondary antibodies or V_{HH} domain fragments of secondary antibodies.

Embodiment 20. The composition of Embodiment 19, wherein the antigen-binding fragments of immunoglobulins target immunoglobulins of the primary antibody's host species.

Embodiment 21. The composition of Embodiment 15, wherein the antigen-binding fragments of immunoglobulins are at a molar ratio with the primary antibody of about a 1:1 to about 3:1, and the a final concentration of the primary antibody is about 0.1 mg/ml to about 1 mg/ml.

Embodiment 22. The composition of Embodiment 15, further comprising a buffer in the mixture with the primary antibody, antigen-binding fragments of immunoglobulins, and the cross-linker.

Embodiment 23. The composition of Embodiment 22, wherein the buffer is phosphate-buffered saline (PBS), phosphate-buffered saline and Tween (PBST), or sodium carbonate.

Embodiment 24. The composition of Embodiment 15, further comprising a denaturant in the mixture with the primary antibody, antigen-binding fragments of immunoglobulins, and the cross-linker.

Embodiment 25. The composition of Embodiment 24, wherein the denaturant is guanidinium chloride at a concentration of about 0.1 M to about 10 M.

Embodiment 26. The composition of Embodiment 15, further comprising a catalyzing agent in the mixture with the primary antibody, antigen-binding fragments of immunoglobulins, and the cross-linker.

Embodiment 27. The composition of Embodiment 26, wherein the catalyzing agent is pyridine or a derivative thereof at a concentration of about 1 mM to about 250 mM.

Embodiment 28. A method of immunolabeling, comprising contacting a composition of a primary antibody, antigen-binding fragments of immunoglobulins, and a cross-linker with biological cells, tissues, or organs to yield a mixture whereby the biological cells, tissues, or organs are immunolabeled.

Embodiment 29. The method of immunolabeling of Embodiment 28, wherein the composition of a primary antibody, antigen-binding fragments of immunoglobulins, and a cross-linker and the biological, cells, tissues, or organs are incubated at a temperature of about 30° C. to about 65° C., about 45° C. to about 60° C., or about 55° C.

Embodiment 30. The method of immunolabeling of Embodiment 28, further comprising adding a buffer and sodium dodecyl sulfate (SDS) to the composition of a primary antibody, antigen-binding fragments of immunoglobulins, and a cross-linker and biological cells, tissues, or organs mixture at about pH of about 6 to about 9, about 7 to about 8, or about 7.4.

Embodiment 31. The method of immunolabeling of Embodiment 30, wherein the buffer is 0.1× to about 10×, 0.5× to about 5×, or about 1× PBS or PBST and the SDS is added at a concentration of about 1% to about 10%, about 2% to about 8%, or about 4%.

Embodiment 32. The method of immunolabeling of Embodiment 28, further comprising a denaturant in the mixture with the primary antibody, antigen-binding fragments of immunoglobulins, and the cross-linker.

Embodiment 33. The method of immunolabeling of Embodiment 32, wherein the denaturant is guanidinium chloride at a concentration of about 0.1 M to about 10 M.

Embodiment 34. The method of immunolabeling of Embodiment 28, further comprising a catalyzing agent in the mixture with the primary antibody, antigen-binding fragments of immunoglobulins, and the cross-linker.

Embodiment 35. The method of immunolabeling of Embodiment 34, wherein the catalyzing agent is pyridine or a derivative thereof at a concentration of about 1 mM to about 250 mM.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A method of stabilizing an antibody, comprising combining the antibody with antigen-binding fragments of immunoglobulins to form a mixture, and adding a cross-linker to the mixture.

2. (canceled)

3. The antibody stabilizing method of claim 1, wherein the antibody is a primary antibody and/or wherein the cross-linker is a homo-multifunctional cross-linker.

4. (canceled)

5. The antibody stabilizing method of claim 1, wherein the cross-linker is diluted to about a 1% to about a 50%, or about a 5% to about a 30%, or about a 10% to about a 20% v/v solution in water and is then added to the antibody or the antibody and the antigen-binding fragments of immunoglobulins mixture at a dilution of about 1:1 to about 1:10, about 1:2 to about 1:8, or about 1:5.

6. The antibody stabilizing method of claim 1, wherein the antigen-binding fragments of immunoglobulins are Fab fragments of secondary antibodies or V_{HH} domain fragments of secondary antibodies or target immunoglobulins of the primary antibody's host species.

7. (canceled)

8. The antibody stabilizing method of claim 1, wherein the antigen-binding fragments of immunoglobulins are incubated with the primary antibody at about a 1:1 to about 3:1 molar ratio for 10 minutes at room temperature, with the primary antibody at a final concentration of about 0.1 to 1 mg/ml.

9. The antibody stabilizing method of claim 1, further comprising providing a buffer in the mixture with the primary antibody and antigen-binding fragments of immunoglobulins or in the mixture of primary antibody, antigen-binding fragments of immunoglobulins, and the cross-linker, wherein the buffer is phosphate-buffered saline (PBS), phosphate-buffered saline and Tween (PBST), or sodium carbonate.

10. (canceled)

11. The antibody stabilizing method of claim 1, further comprising providing a denaturant in the mixture with the primary antibody and antigen-binding fragments of immunoglobulins or in the mixture with the primary antibody, antigen-binding fragments of immunoglobulins, and the cross-linker, wherein the denaturant is guanidinium chloride at a concentration of about 0.1 M to about 10 M.

12. (canceled)

13. The antibody stabilizing method of claim 1, further comprising providing a catalyzing agent in the mixture with the primary antibody and antigen-binding fragments of immunoglobulins or in the mixture of primary antibody, antigen-binding fragments of immunoglobulins, and the cross-linker, wherein the catalyzing agent is pyridine or a derivative thereof at a concentration of about 1 mM to about 250 mM.

14. (canceled)

15. An antibody composition comprising a primary antibody, antigen-binding fragments of immunoglobulins, and a cross-linker.

16. The composition of claim 15, wherein the cross-linker is a homo-multifunctional cross-linker.

17. (canceled)

18. The composition of claim 15, wherein the cross-linker diluted to about a 1% to about 50%, or about a 5% to about a 30%, or about a 10% to about a 20% v/v solution in water is and the antigen-binding fragments of immunoglobulins at a 1:5 dilution.

19. The composition of claim 15, wherein the antigen-binding fragments of immunoglobulins are Fab fragments of secondary antibodies or V_{HH} domain fragments of secondary antibodies and target immunoglobulins of the primary antibody's host species.

20. (canceled)

21. The composition of claim 15, wherein the antigen-binding fragments of immunoglobulins are at a molar ratio with the primary antibody of about a 1:1 to about 3:1, and the a final concentration of the primary antibody is about 0.1 mg/ml to about 1 mg/ml.

22. The composition of claim 15, further comprising a buffer in the mixture with the primary antibody, antigen-binding fragments of immunoglobulins, and the cross-linker, wherein the buffer is phosphate-buffered saline (PBS), phosphate-buffered saline and Tween (PBST), or sodium carbonate.

23. (canceled)

24. The composition of claim 15, further comprising a denaturant in the mixture with the primary antibody, antigen-binding fragments of immunoglobulins, and the cross-linker, wherein the denaturant is guanidinium chloride at a concentration of about 0.1 M to about 10 M.

25. (canceled)

26. The composition of claim 15, further comprising a catalyzing agent in the mixture with the primary antibody, antigen-binding fragments of immunoglobulins, and the cross-linker, wherein the catalyzing agent is pyridine or a derivative thereof at a concentration of about 1 mM to about 250 mM.

27. (canceled)

28. A method of immunolabeling, comprising contacting a composition of a primary antibody, antigen-binding fragments of immunoglobulins, and a cross-linker with biological cells, tissues, or organs to yield a mixture whereby the biological cells, tissues, or organs are immunolabeled, wherein the composition of a primary antibody, antigen-binding fragments of immunoglobulins, and a cross-linker and the biological, cells, tissues, or organs are incubated at a temperature of about 30° C. to about 65° C., about 45° C. to about 60° C., or about 55° C.

29. (canceled)

30. The method of immunolabeling of claim 28, further comprising adding a buffer and sodium dodecyl sulfate (SDS) to the composition of a primary antibody, antigen-binding fragments of immunoglobulins, and a cross-linker and biological cells, tissues, or organs mixture at about pH of about 6 to about 9, about 7 to about 8, or about 7.4, wherein the buffer is 0.1× to about 10×, 0.5× to about 5×, or about 1× PBS or PBST and the SDS is added at a concentration of about 1% to about 10%, about 2% to about 8%, or about 4%.

31. (canceled)

32. The method of immunolabeling of claim 28, further comprising a denaturant in the mixture with the primary antibody, antigen-binding fragments of immunoglobulins, and the cross-linker, wherein the denaturant is guanidinium chloride at a concentration of about 0.1 M to about 10 M.

33. (canceled)

34. The method of immunolabeling of claim 28, further comprising a catalyzing agent in the mixture with the primary antibody, antigen-binding fragments of immunoglobulins, and the cross-linker, wherein the catalyzing agent is pyridine or a derivative thereof at a concentration of about 1 mM to about 250 mM.

35. (canceled)
